(19) United States
(12) Patent Application Publication
(10) Pub. No.: US 2024/0426824 A1
(43) Pub. Date: Dec. 26, 2024

(54) METHODS RELATING TO TREATMENT OF ACUTE MYELOID LEUKEMIA

(71) Applicants: Stefan E. HUBNER, Houston, TX (US); BOARD OF REGENTS, THE UNIVERSITY OF TEXAS SYSTEM, Austin, TX (US)

(72) Inventors: Stefan E. HUBNER, Houston, TX (US); Brandon D. BROWN, Houston, TX (US); Steven M. KORNBLAU, Houston, TX (US)

(21) Appl. No.: 18/707,277

(22) PCT Filed: Nov. 2, 2022
(86) PCT No.: PCT/US2022/079172

§ 371 Date: May 3, 2024

Related U.S. Application Data
(60) Provisional application No. 63/275,257, filed on Nov. 3, 2021.

(51) Int. Cl. G01N 33/574

(52) U.S. Cl. CPC G01N 33/57426; G01N 33/57492; G01N 2800/52

(57) ABSTRACT

A method of determining the likelihood that a subject with acute myeloid leukemia (AML) will respond to a treatment comprising a BCL-2 inhibitor is provided. The method comprises detecting the levels of protein biomarkers in abiological sample obtained from a human subject. Subjects determined to have a high likelihood of responding to a BCL-2 inhibitor treatment can be selected to continue or initiate the treatment, while subjects determined to have a low likelihood of responding can discontinue treatment and/or be administered an alternative therapy. Methods of treating AML patients are also provided, as are kits and systems for biomarker detection, recordation, and responder status determination.

### RELATED APPLICATIONS

This application claims the benefit of and priority to U.S. Provisional Application No. 63/275,257, filed on Nov. 3, 2021, the entire content of which is herein incorporated by reference for all purposes.

### BACKGROUND

Acute myeloid leukemia (AML), the most common acute leukemia in adults, is a heterogeneous disease with variable responses to induction therapy and survival outcomes. Disease incidence rises with age, with the median age at diagnosis being 68 years. Toxicity associated with historical chemotherapeutic regimens have limited available options for older, less fit patients. Less intensive treatment regimens such as the B-cell lymphoma-2 (BCL-2) inhibitor venetoclax (VTX), combined with a hypomethylating agent or low-dose cytarabine have been well tolerated and effective. However, resistance and relapse still occur in the majority of patients. Also, although alterations in apoptosis-related gene products such as MCL1 and BCL XL are noted at relapse, identification of prognostic features remain unknown. Notably, BCL-2 expression at diagnosis has not been shown to correlate with response to treatment, suggesting that resistance may be acquired by dysregulation of other BCL2 family proteins.

There is therefore a need for new prognostic markers that could guide the use of VTX in patients that have a favorable likelihood of response and/or inform post-remission therapy in patients that have a high likelihood of relapse. The present disclosure addresses this need and provides other advantages as well.

### BRIEF SUMMARY

In one aspect, the present disclosure provides a method of determining the likelihood that a subject with acute myeloid leukemia (AML) will respond to a therapy comprising the administration of a BCL-2 inhibitor, the method comprising detecting protein levels of one or more AML therapy response biomarkers in a biological sample comprising AML cells obtained from the subject, wherein the one or more AML therapy response biomarkers comprises a biomarker listed in Table 3. In some embodiments, the AML therapy response biomarkers comprises one or more biomarkers listed in Table 9. In some embodiments, 2, 3, 4, 5, 6, 7, 8, 9, 10, or more AML therapy response biomarkers are detected in the biological sample. In some embodiments, the AML therapy biomarkers comprise one or more of CDKN1B.pT198, MSH6, ASH2L, MDM2.pS166, or ITGA2.

In some embodiments, the method further comprises calculating a response score based on the detected protein levels of the AML therapy response biomarkers, wherein the response score corresponds to the likelihood that the subject will respond to the therapy. In some embodiments, the subject is determined to have a high likelihood of responding to the therapy. In some embodiments, the method further comprises administering the therapy to the subject. In some embodiments, the subject is determined to have a low likelihood of responding to the therapy. In some embodiments, the method further comprises administering an alternative therapy to the subject that does not comprise the BCL-2 inhibitor. In some embodiments, the BCL-2 inhibitor is venetoclax. In some embodiments, a response to the therapy comprises an increase in remission duration. In some embodiments, a response to the therapy comprises an increase in overall survival. In some embodiments, the biological sample is a blood sample or a bone marrow sample. In some embodiments, the protein levels of the one or more AML therapy response biomarkers are measured using one or more antibodies that specifically bind to the one or more biomarkers.

In another aspect, the present disclosure provides a method of generating a report containing information on the likelihood that a subject with AML will respond to a therapy comprising a BCL-2 inhibitor, comprising: detecting in a biological sample obtained from the subject the protein levels of one or more AML therapy response biomarkers; and, generating the report, wherein the one or more AML therapy response biomarkers comprise one or more biomarkers listed in Table 3, Table 4, Table 6, Table 7, or Table 8, and wherein the report is useful for determining the likelihood that the subject will respond to the therapy. In some embodiments, the one or more AML therapy response biomarkers comprise one or more of the biomarkers listed in Table 9.

In some embodiments, the one or more AML therapy response biomarkers comprise at least one of CDKN1B.pT198, MSH6, ASH2L, MDM2.pS166, or ITGA2.

In another aspect, the present disclosure provides a system for determining the likelihood that a subject with AML will respond to a therapy comprising a BCL-2 inhibitor, comprising a station for analyzing a biological sample comprising AML cells obtained from the subject to measure protein levels of one or more AML therapy response biomarkers in the sample, wherein the one or more AML therapy response biomarkers comprise one or more biomarkers listed in Table 3, Table 4, Table 6, Table 7, or Table 8.

In some embodiments, the AML therapy response biomarkers comprise at least one of the biomarkers listed in Table 9. In some embodiments, the one or more AML therapy response biomarkers comprise at least one of CDKN1B.pT198, MSH6, ASH2L, MDM2.pS166, or ITGA2. In some embodiments, the system further comprises a station for generating a report containing information on results of the analyzing.

In another aspect, the present disclosure provides a method of treating a subject with acute myeloid leukemia (AML), the method comprising administering to the subject a therapeutically effective amount of a BCL-2 inhibitor to a subject having differential levels of one or more AML response biomarkers in a biological sample from the subject as compared to a control, wherein the one or more biomarkers comprise at least one of CDKN1B.pT198, MSH6, ASH2L, MDM2.pS166, or ITGA2, and/or any one or more of biomarkers listed in Table 3, Table 4, Table 6, Table 7, Table 8, or Table 9.

In another aspect, the present disclosure provides a method of treating a subject with acute myeloid leukemia (AML), the method comprising administering to the subject a therapeutically effective amount of a BCL-2 inhibitor, wherein the subject has been identified as a likely responder to the BCL-2 inhibitor based on a detection of the protein levels of one or more AML therapy response biomarkers in a biological sample comprising AML cells obtained from the subject, wherein the identification of the subject as a likely responder is based on a difference in the one or more protein levels relative to protein levels of the one or more AML therapy response biomarkers in a biological sample from a healthy individual without AML, and wherein the one or more AML therapy response biomarkers comprises a biomarker listed in Table 3, Table 4, Table 6, Table 7, or Table 8. In some embodiments, the AML therapy response biomarkers comprise at least one of the biomarkers listed in Table 9. In some embodiments, 2, 3, 4, 5, 6, 7, 8, 9, 10, or more AML therapy response biomarkers are detected in the biological sample. In some embodiments, the AML therapy response biomarkers comprise at least one of CDKN1B.pT198, MSH6, ASH2L, MDM2.pS166, or ITGA2.

In some embodiments, the identification of the subject as a likely responder comprises the calculation of a response score based on the detected protein levels of the AML therapy response biomarkers, wherein the response score corresponds to the likelihood that the subject will respond to the therapy. In some embodiments, the BCL-2 inhibitor is venetoclax. In some embodiments, the administration of the therapy leads to an increase in remission duration. In some embodiments, the administration of the therapy leads to an increase in overall survival. In some embodiments, the biological sample is a blood sample or a bone marrow sample. In some embodiments, wherein the protein levels of the one or more AML therapy response biomarkers are measured using one or more antibodies that specifically bind to the one or more biomarkers. In some embodiments, the protein levels are measured using an ELISA assay.

In another aspect, the present disclosure comprises a method of generating a report containing information on the likelihood that a subject with AML will not respond to a therapy comprising a BCL-2 inhibitor, comprising detecting in a biological sample obtained from the subject the protein levels of one or more AML therapy response biomarkers; and, generating the report, wherein the one or more AML therapy response biomarkers comprise one or more biomarkers listed in Table 3, Table 4, Table 6, Table 7, or Table 8, and wherein the report is useful for determining the likelihood that the subject will not respond to the therapy. In some embodiments, the AML therapy response biomarkers comprise one or more biomarkers listed in Table 9. In some embodiments, the one or more AML therapy response biomarkers comprise at least one of CDKN1B.pT198, MSH6, ASH2L, MDM2.pS166, or ITGA2.

### BRIEF DESCRIPTION OF THE DRAWINGS

FIGS. 1A-1B. Kaplan-Meier survival curves for overall survival (FIG. 1A) and remission duration (FIG. 1B) in two cohorts by hierarchical clustering of 44-protein classifier correlated with remission duration.

FIG. 2. CART modeling of the VTX treated cohort identified 3 proteins—SPI1, NOTCH1.cle, and PTPN12—that predicted cluster membership with a computed accuracy of 94.3% (misclassification error 6%). The values shown in the classifier for each of the 3 proteins is a cut-off for expression. Expression is given as a value relative to the normal control with the normal control set to 0. Specifically, a numerical value on a log 2 scale was utilized, with a value of 1 indicating 1 log base 2 higher than median, a value of 2 indicating 2 log base 2 higher (i.e., 4 fold higher), a value of 3 indicating 3 log base 2 higher (i.e., 8 fold higher), etc.

FIG. 3A-3B. FIG. 3A depicts overall survival (OS) (n=138), and FIG. 3B depicts remission duration (RD) (n=90) for the three clusters of patients identified using the 36-protein classifier (Table 4).

FIGS. 4A-4B. FIG. 4A depicts protein expression in VTX-resistant cell lines (MOLM-13-VTX-res and OCI-3) characterized by increased baseline expression of potential protein targets relative to MOLM-13 VTX-sensitive cell line. FIG. 4B depicts 24 hr VTX treatment attenuating CBL expression in MOLM-13 VTX-sensitive cell line while increasing CBL expression in VTX-resistant OCI-3 cell line. Similarly, 24 hr VTX treatment reduces SYK expression in MOLM-13 VTX-sensitive line while inducing SYK expression in two VTX-resistant cell lines.

FIG. 5 depicts a random forests returned model-specific set of proteins with high SHapley Additive exPlanations (SHAP) values correlated to having the strongest contribution to the predictive value of the model.

### DETAILED DESCRIPTION

Provided herein are methods and compositions for determining the likelihood that a subject with acute myeloid leukemia (AML) will respond to a BCL-2 inhibitor such as venetoclax. In particular, the present disclosure involves methods for measuring the protein levels of one or more biomarkers in order to determine whether a subject has a high or low likelihood of responding to the inhibitor. Responding can involve, inter alia, an increase in overall survival, an increased duration of remission, a decreased likelihood of relapse, or any other measure indicating that the BCL-2 inhibitor is acting to reduce, eliminate, slow, attenuate, or otherwise negatively affect the growth, proliferation, and/or survival of AML cells in the subject. Subjects that are determined to have a high likelihood of responding to an inhibitor can, e.g., be treated using an inhibitor such as venetoclax, whereas subjects determined to have a low likelihood of responding can be treated using an alternative therapeutic approach, such as an approach that does not include venetoclax, or an approach including venetoclax in combination with another agent.

### A. Subjects and Samples

The present methods and compositions can be used to determine whether a subject with acute myeloid leukemia (AML) is likely to respond to a treatment comprising the administration of a BCL-2 inhibitor such as venetoclax. In various embodiments, the subject may be an adult of any age, a child, or an adolescent. The subject may be male or female. In particular embodiments, the subject is a human.

“Acute myeloid leukemia” or “AML” refers to a type of blood cancer that can be present in the bone marrow, blood, liver, spleen, and other organs. AML can also be referred to by other names such as “acute myelocytic leukemia,” acute myelogenous leukemia,” “acute granulocytic leukemia,” or “acute non-lymphocytic leukemia.” Any type or form of AML can be treated using the present methods, including subtypes M0, M1, M2, M3, M4, M5, M6, and M7, per the French-American-British classification system, and can involve AML originating in different blood cell types such as white blood cells, red blood cells, and platelets. Also encompassed are AMLs with genetic abnormalities including, e.g., AML with a translocation between chromosomes 8 and 21 [t(8;21)], AML with a translocation or inversion in chromosome 16 [t(16;16) or inv(16)], APL with the PML-RARA fusion gene, AML with a translocation between chromosomes 9 and 11 [t(9;11)], AML with a translocation between chromosomes 6 and 9 [t(6:9)], AML with a translocation or inversion in chromosome 3 [t(3;3) or inv(3)], AML (megakaryoblastic) with a translocation between chromosomes 1 and 22 [t(1:22)], AML with the BCR-ABL1 (BCR-ABL) fusion gene, AML with a mutated NPM1 gene, AML with biallelic mutations of the CEBPA gene, and AML with a mutated RUNX1 gene. The disclosure also comprises treatment of AML with myelodysplasia-related changes, AML related to previous chemotherapy or radiation, AML not otherwise specified, AML with minimal differentiation (FAB MO), AML without maturation (FAB M1), AML with maturation (FAB M2), acute myelomonocytic leukemia (FAB M4), Acute monoblastic/monocytic leukemia (FAB M5), pure erythroid leukemia (FAB M6), acute megakaryoblastic leukemia (FAB M7), acute basophilic leukemia, acute panmyelosis with fibrosis, myeloid sarcoma (also known as granulocytic sarcoma or chloroma), and myeloid proliferations related to Down syndrome.

The subject may have one or more symptoms of AML. A non-limiting list of possible symptoms includes loss of appetite, weight loss, fatigue, weakness, fever, feeling cold, dizzy, or lightheaded, headaches, pale skin, shortness of breath, bruises or other red or purple spots on the skin, excess bleeding, nosebleeds, bleeding gums, weakness in one side of the body, slurred speech, confusion, sleepiness, blurry vision, loss of vision, chest pain, bone pain, joint pain, swelling in the abdomen, nausea, facial numbness, loss of balance, seizures, enlarged lymph nodes, anemia, leukopenia, neutropenia, thrombocytopenia, and others. The symptoms can be mild, moderate, or severe. A diagnosis of AML can be based on, e.g., medical history, physical exam, or lab test, e.g., as performed on a blood sample, bone marrow sample (e.g., aspiration or biopsy), or cerebrospinal fluid sample. Lab tests can comprise, e.g., blood cell counts, coagulation tests, cell analysis by microscope, cytochemical analysis, flow cytometry, immunohistochemistry, cytogenetic test, FISH, PCR, imaging tests such as X-rays, CT scan, PET scan, MRI, ultrasound, and others.

A “response” to a BCL-2 inhibitor can refer to any lasting, detectable improvement in any symptom of AML (e.g., any symptom as described elsewhere herein) in the subject. A patient or subject showing a response to a therapy means that the patient is a “responder” or is “responsive” to the treatment. In particular embodiments, a determination that a patient is a “responder” to a BCL-2 inhibitor means that the patient shows a detectable improvement in survival (i.e., the patient survives longer than an otherwise equivalent patient not receiving a BCL-2 inhibitor-comprising therapy), remission duration (i.e., the patient is in remission for a longer period that in an otherwise equivalent patient not receiving a BCL-2 inhibitor-comprising therapy), and/or likelihood of relapse. An AML patient that is determined to likely be a responder to a BCL-2 inhibitor using the herein-described methods is considered a “candidate” for a therapy comprising a BCL-2 inhibitor.

In some embodiments, the subject is being treated with a therapy for AML. In some embodiments, the subject is being treated with a BCL-2 inhibitor (e.g., Venetoclax (VEN)). In some embodiments, the therapy can include a BCL-2 inhibitor (e.g., Venetoclax (VEN)), a hypomethylating agent (HMA), conventional chemotherapy (CC), and/or an additional or alternative therapy. Such therapies are discussed below in Section E in more detail.

To assess the “BCL-2 inhibitor responder status” of the subject (i.e., whether the subject has a high or low likelihood of responding to the inhibitor, as discussed below in Section D), a biological sample is obtained from the subject. In some embodiments, the biological sample is a blood sample, such as serum or whole blood. In some embodiments, the biological sample is a bone marrow sample, e.g., a bone marrow aspirate or biopsy. Generally, any sample that comprises AML cells (i.e., leukemic cells) can be used, including cell or tissue samples (e.g., biopsies or swabs) from one or more organs that can be infiltrated by AML cells such as the spleen, liver, gums, skin, CNS, testicles, and others. Other suitable samples include urine, ascites, seminal fluid, vaginal secretions, cerebrospinal fluid (CSF), synovial fluid, pleural fluid (pleural lavage), pericardial fluid, peritoneal fluid, amniotic fluid, saliva, nasal fluid, otic fluid, gastric fluid, breast milk, amniotic fluid, bile, gastric juice, lymph, mucus, pericardial fluid, peritoneal fluid, pleural fluid, pus, saliva, sebum, serous fluid, sputum, sweat, tears, and others. The sample can be obtained from the subject using conventional techniques known in the art.

### B. Selection of Biomarkers

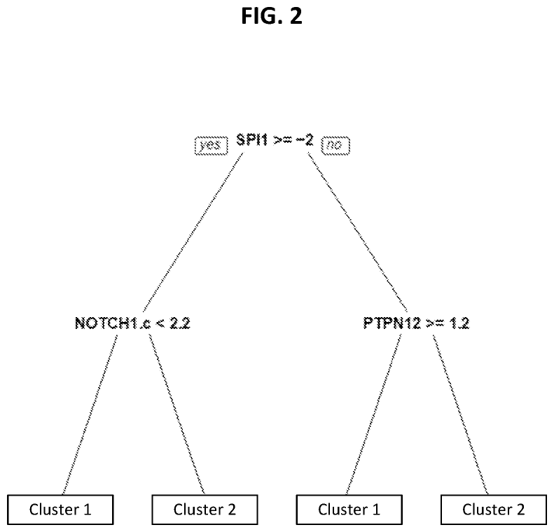

The likelihood of an AML patient responding to a BCL-2 inhibitor is determined by detecting in a biological sample levels of “AML therapy response” biomarkers, also referred to herein as “AML-response” or “AML therapy” biomarkers. As used herein, a “biomarker” refers to a molecule whose level in a biological sample, e.g., a blood sample is correlated with a high or low likelihood of responding to a BCL-2 inhibitor (i.e., their “responder status”). In particular embodiments, the levels are protein levels within the sample. The protein levels of each of the biomarkers need not be correlated with the responder status in all subjects; rather, a correlation will exist at the population level, such that the level is sufficiently correlated within the overall population of individuals with AML that it can be combined with the levels of other biomarkers, in any of a number of ways, as described elsewhere herein, and used to determine the responder status. The values used for the measured levels of the individual biomarkers can be determined in any of a number of ways, including direct readouts from relevant instruments or assay systems, e.g., using means known to those of skill in the art. In some embodiments, the readout values of the biomarkers are compared to the readout value of a reference or control. For example, the reference or control can be a protein whose level does not vary according to responder status and whose level is measured at the same time as the biomarkers. In another example, the reference or control is a known amount of an isolated protein of the biomarker(s) being assessed in the biological sample. Other possible controls include, inter alia, a reference level of the measured biomarker protein that is representative of a level from a healthy individual without AML, or a level calculated from a population of healthy individuals without AML, or a level measured from non-AML cells of the patient. In particular embodiments, the control is a level of expression of the biomarker genes in otherwise equivalent non-AML blast cells. For example, because the majority of AML blasts are CD34+, in some embodiments, the control cells are CD34+ bone marrow cells from a subject without AML. Such control CD34+ cells can be obtained, e.g., from commercial sources. In some embodiments, the median expression of the biomarker protein and associated range is obtained from a plurality of such control cells, e.g., samples from 10 different individuals. The expression level of the biomarker protein in the AML cells is then compared to the expression level of the biomarker protein in the control cells to determine if the AML-associated expression is above or below normal and/or if it is within the normal range. The relative expression can be defined using a numerical value on a log 2 scale, e.g., with a value of 1 corresponding to 1 log base 2 higher than median, a value of 2 corresponding to 2 log base 2 higher than median (i.e., 4-fold higher), a value of 3 corresponding to 3 log base 2 higher than median (e.g., 8-fold higher), etc. Such values are used, e.g., to indicate the expression of the AML cells relative to control in FIG. 2. In some embodiments, a standardized amount of the purified protein of interest (i.e. the biomarker protein) can be used as a reference, e.g., in a set of serial dilutions, and the expression in cells from the AML patient is compared to expression in the controls to determine quantified amounts, e.g., in picograms.

The biomarkers used in the present methods correspond to proteins whose levels in biological samples comprising AML, e.g., blood samples or bone marrow samples, from the subject correlate with the likelihood of the subject responding to a therapy comprising a BCL-2 inhibitor. The level of the individual biomarkers can be elevated or depressed in responders relative to the level in individuals without AML or in non-responders. What is important is that the level of the biomarker is positively or inversely correlated with the likelihood of responding to the BCL-2 inhibitor, allowing a treatment decision to be made with respect to the subject (e.g., to administer a BCL-2 inhibitor or to select an alternative treatment).

The term “correlating” generally refers to determining a relationship between one random variable with another. In various embodiments, correlating a given biomarker level with a high or low likelihood of responding to a BCL-2 inhibitor comprises determining the presence, absence or amount of at least one biomarker in a subject with the same outcome. In some embodiments, a set of biomarker levels, absences or presences is correlated to a particular outcome, using receiver operating characteristic (ROC) curves. In some embodiments, a set of biomarker levels, absences or presences is correlated to a particular outcome, using SHAP values. In some embodiments, a set of biomarker levels, absences or presences is correlated to a particular outcome, using random forests analysis. For example, random forests is an ensemble learning method for classification, regression and other tasks that operates by constructing a multitude of decision trees at training time. Random forests combine predictions from many different decision trees to create a robust classifier that has enhanced predictive performance relative to individual trees. In certain embodiments, as described in this disclosure, random forests were generated using the expression of proteins as measured by RPPA to algorithmically predict cluster assignment.

In some embodiments, the biomarkers comprise one or more of the proteins listed in Table 1. In particular embodiments, the biomarkers comprise one or more of the proteins in Table 1 corresponding to proteins correlated with remission duration (bolded) and/or overall survival (underlined) in AML patients treated with a BCL-2 inhibitor such as venetoclax. In particular embodiments, the biomarkers comprise one or more of SPI1 (also referred to as PU.1), NOTCH.cle, or PTPN12. SPI1 (Spi-1 proto oncogene; see, e.g., PubChem Gene ID No. 6688, the entire disclosure of which is herein incorporated by reference) refers to a gene encoding an ETS-domain transcription factor called PU.1 (see, e.g., UniProt ID P17947, the entire disclosure of which is herein incorporated by reference). NOTCH.cle refers to a cleaved form of the Notch receptor (see, e.g., UniProt P46531, the entire disclosure of which is herein incorporated by reference). In particular, NOTCH.cle corresponds to the product of the activating cleavage event by gamma secretase. This cleavage product, also referred to as NOTCH1 and ICN1, can translocate from the cytosol into the nuclease where it can affect gene expression. PTPN12 (protein tyrosine phosphatase non-receptor type 12; see, e.g., NCBI Gene ID 5782, the entire disclosure of which is herein incorporated by reference).

In some embodiments, the biomarkers comprise one or more of the proteins listed in Table 3. In particular embodiments, the biomarkers comprise one or more of the proteins in Table 4 corresponding to proteins correlated with overall survival in AML patients treated with a BCL-2 inhibitor such as Venetoclax. In some embodiments, the biomarkers comprise one or more of the proteins listed in Table 9. In some embodiments, the biomarkers comprise one or more of CDKN1B.pT198, MSH6, ASH2L, MDM2.pS166, or ITGA2.

Other biomarkers can also be used, e.g., in place of or in addition to any one or more of SPI1, NOTCH.cle, or PTPN12. For example, in some embodiments, biomarkers used in the methods include, but are not limited to, any one or more of the protein biomarkers listed in Table 1. In particular embodiments, the one or more protein biomarkers comprise proteins listed in Table 1 that are underlined and/or in bold. It will be appreciated that any one or more of the biomarkers, or sets of biomarkers, listed in Table 1, can be used alone or in any combination. Any number of biomarkers can be assessed in the methods, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 50, 60, 70, 80, 90, 100 or more biomarkers. In some embodiments, the value of a biomarker listed in Table 1 is within a yes/no fit in a cluster 1 or 2 profile, as described in the Examples, with the overall cluster designation based on the summary of the expression of SPI1, NOTCH.cle, and PTPN12 (i.e., whether they are above or below the cutoff for each protein), i.e., the result of the two decision nodes indicated in FIG. 2.

Alternatively, in some embodiments, biomarkers used in the methods include, but are not limited to, any one or more of the protein biomarkers listed in Table 3. In particular embodiments, any of the one or more biomarkers listed in Table 4 can be used. In particular embodiments, any of the one or more biomarkers listed in Table 9 can be used. It will be appreciated that any one or more of the biomarkers, or sets of biomarkers, listed in Table 3, Table 4, and/or Table 9 can be used alone or in any combination. Any number of biomarkers can be assessed in the methods, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 50, 60, 70, 80, 90, 100 or more biomarkers.

Additional AML response protein biomarkers can be assessed and identified using any standard analysis method or metric, e.g., by analyzing data from biological samples taken from subjects with AML and with a known responder or non-responder status to a BCL-2 inhibitor, as described in more detail elsewhere herein and as illustrated, e.g., in the Examples. For example, in some embodiments, differences in protein expression levels, e.g., as assessed using reverse phase protein array (RPPA), are analyzed by Pearson correlation to identify significant protein-protein correlations. In some embodiments, survival curves are generated by the Kaplan-Meier method and the survival data analyzed by multivariate cox regression model. In some embodiments, protein expression signatures are identified by hierarchical clustering, and predictive models of protein classifiers are determined by classification and regression trees (CART) analysis.

### C. Detecting Biomarker Levels

The levels of the biomarkers in the sample can be assessed in any of a number of ways. In particular embodiments, the sample, e.g., blood sample or bone marrow sample, is prepared by fractionated to allow enrichment of leukemia blast cells, and the protein biomarker levels are determined in lysates from the fraction comprising the leukemia blast cells. In some instances, the levels of the biomarkers are determined using antibody-based assays, e.g., using antibody microarrays. In some embodiments, the protein levels are measured using reverse phase protein array (RPPA) such described, for example, in Tibes et al. 2006 and Kornblau et al. 2011. In particular embodiments, the protein levels are measured using an ELISA-based assay. In some embodiments, an internal control is used, e.g., a reference protein whose level is known to not vary in correlation with the likelihood of responding to a BCL-2 inhibitor in AML patients. In some embodiments, one or more of the herein-described biomarkers are analyzed together with one or more clinical features of the subject or results of other molecular or cytological analyses (see, e.g., Table 2 or Table 5).

The measurement of biomarker protein levels can be assessed using routine techniques such as immunoassays, two-dimensional gel electrophoresis, and quantitative mass spectrometry that are known to those skilled in the art. Protein quantification techniques are generally described in “Strategies for Protein Quantitation,” Principles of Proteomics, 2nd Edition, R. Twyman, ed., Garland Science, 2013. In some embodiments, protein expression or stability is detected by immunoassay, such as but not limited to enzyme immunoassays (EIA) such as enzyme multiplied immunoassay technique (EMIT), enzyme-linked immunosorbent assay (ELISA), IgM antibody capture ELISA (MAC ELISA), and microparticle enzyme immunoassay (MEIA); capillary electrophoresis immunoassays (CEIA); radioimmunoassays (RIA); immunoradiometric assays (IRMA); immunofluorescence (IF); fluorescence polarization immunoassays (FPIA); and chemiluminescence assays (CL). If desired, such immunoassays can be automated. Immunoassays can also be used in conjunction with laser induced fluorescence (see, e.g., Schmalzing et al., Electrophoresis, 18:2184-93 (1997); Bao, J. Chromatogr. B. Biomed. Sci., 699:463-80 (1997)).

In some embodiments, replicates (e.g., duplicates, triplicates) of any of the herein-described assays may be run for each sample in order to gain a higher level of confidence in the data. Replicate values can be averaged, and standard deviations can be calculated.

In some embodiments, the detection is carried out in whole or in part using an integrated system, as described elsewhere herein, which may also comprise a computer system as described elsewhere herein.

In some embodiments, as described elsewhere herein, random forests were generated using the expression of proteins as measured by RPPA to algorithmically predict cluster assignment. Prospective validation may be used to extend the analysis to other protein detection methods. For a given prospective validation sample (i.e. classifier protein set), the relevant protein expression may be measured via both RPPA and the selected protein detection method (e.g., ELISA). RPPA protein expression values for each relevant protein can be inputted into the random forest model to return a predicted classification label for the sample. This classification may then be the label for a sample in a new random forest model that uses the selected expression detection method of the same proteins to predict classifications. As such, the new random forest will most accurately learn the transduction function (“cutoff values”) necessary to convert the protein expression values measured using the selected expression detection method to the appropriate cluster classification.

In some embodiments, as described elsewhere herein, random forests were generated using the expression of proteins as measured by RPPA to algorithmically predict cluster assignment. Prospective validation may be used to extend the analysis to other protein detection methods. For a given prospective validation sample (i.e. classifier protein set), the relevant protein expression may be measured via both RPPA and the selected protein detection method (e.g., ELISA). RPPA protein expression values for each relevant protein can be inputted into the random forest model to return a predicted classification label for the sample. This classification may then be the label for a sample in a new random forest model that uses the selected expression detection method of the same proteins to predict classifications. As such, the new random forest will most accurately learn the transduction function (“cutoff values”) necessary to convert the protein expression values measured using the selected expression detection method to the appropriate cluster classification.

### D. Determining Aml Responder Status

To determine the likelihood of responding to a BCL-2 inhibitor (i.e., the “BCL-2 inhibitor responder status”) in an individual (i.e., a subject or AML patient), the measured biomarker levels in a sample obtained from the individual are generally compared to reference levels, e.g., levels taken from a healthy individual without AML. The reference control levels can be measured at the same time as the biomarker levels, i.e., using the same sample, or can be a level determined based on previous measurements.

Thus, in one aspect, provided herein is a method of determining the likelihood of responding to a BCL-2 inhibitor in a subject with AML comprising, consisting essentially of, or consisting of: detecting differential levels of one or more AML response biomarkers as disclosed herein in an AML cell-comprising biological sample obtained from the subject as compared to a control, wherein the one or more biomarkers comprise one or more of STI1, Notch.cle, PTPN12, and/or any of the proteins listed in Table 1, and in particular proteins listed in Table 1 that are underlined and/or in bold.

Thus, in another aspect, provided herein is a method of determining the likelihood of responding to a BCL-2 inhibitor in a subject with AML comprising, consisting essentially of, or consisting of: detecting differential levels of one or more AML response biomarkers as disclosed herein in an AML cell-comprising biological sample obtained from the subject as compared to a control, wherein the one or more biomarkers comprise one or more of the proteins listed in Table 3, Table 4, or Table 9.

In particular embodiments, the method comprises: providing a biological sample comprising AML cells from the subject (e.g., blood or bone marrow); detecting the one or more AML response biomarkers in the sample, and comparing the levels of the one or more biomarkers to a control (i.e. reference protein), wherein the determination of the responder status in the subject is made according to the decision tree shown in FIG. 2. For example, in some embodiments, the expression of SPI1 (i.e. PU.1) is determined relative to a control level, wherein the expression is given as a value relative to a normal control with the control value set to 0. In some embodiments, the relative expression level of SPI1 is determined to be greater than or equal to −2, and the relative expression level of NOTCH1.cle (i.e., the expression value relative to a normal NOTCH.cle control value set to 0) is determined to be less than 2.2. In such embodiments, the subject is determined to have a low likelihood of responding to the BCL-2 inhibitor. In some embodiments, the relative expression level of SPI1 is determined to be less than −2, and the relative expression level of PTPN12 is determined to be greater than or equal to 1.2 2.2. In such embodiments, the subject is determined to have a low likelihood of responding to the BCL-2 inhibitor. In some embodiments, the relative expression level of SPI1 is determined to be greater than or equal to −2, and the relative expression level of NOTCH1.cle is determined to be greater than 2.2. In such embodiments, the subject is determined to have a high likelihood of responding to the BCL-2 inhibitor. In some embodiments, the relative expression level of SPI1 is determined to be less than −2, and the relative expression level of PTPN12 is determined to be less than 1.2. In such embodiments, the subject is determined to have a high likelihood of responding to the BCL-2 inhibitor.

When using multiple biomarkers, it is not necessary that all of the biomarkers are elevated or depressed relative to control levels in a biological sample from a given subject to give rise to a determination of the subject's responder status. For example, for a given biomarker level there can be some overlap between individuals falling into different responder categories. However, collectively the combined levels for all of the biomarkers included in the assay gives rise to a score or other calculated value that indicates a high probability of, e.g., responding to the BCL-2 inhibitor.

In some embodiments, the levels of the selected biomarkers are quantified and compared to one or more preselected or threshold levels. Threshold values can be selected that provide an ability to predict the likelihood of responding to a BCL-2 inhibitor. Such threshold values can be established, e.g., by calculating receiver operating characteristic (ROC) curves using a first AML patient population that responds to a BCL-2 inhibitor and a second AML patient population that does not respond to a BCL-2 inhibitor.

In some embodiments, a classifier is generated (also referred to as training) for use in the methods of determining the likelihood of responding to a BCL-2 inhibitor in a subject with AML. As used herein, the terms “classifier” and “predictor” are used interchangeably and refer to a mathematical function that uses the values of the signature (e.g. protein levels from a defined set of biomarkers) and a pre-determined coefficient for each signature component to generate scores for a given observation or individual patient for the purpose of assignment to a category. A classifier is linear if scores are a function of summed signature values weighted by a set of coefficients. Furthermore, a classifier is probabilistic if the function of signature values generates a probability, a value between 0 and 1.0 (or 0 and 100%) quantifying the likelihood that a subject or observation belongs to a particular category or will have a particular outcome, respectively. Probit regression and logistic regression are examples of probabilistic linear classifiers.

A classifier, including a linear classifier, may be obtained by a procedure known as training, which consists of using a set of data containing observations with known category membership (e.g., subjects responding favorably, unfavorably, or very unfavorably to a BCL-2 inhibitor). Specifically, training seeks to find the optimal coefficient for each component of a given signature, where the optimal result is determined by the highest classification accuracy. In some embodiments, a unique classifier may be developed and trained with respect to a particular platform upon which the signature is measured.

For example, classifiers that use host protein biomarker levels can be generated from a training set of samples obtained from AML patients having a known responder status. Measurements of many proteins can be obtained, e.g., by RPPA as described elsewhere herein. The measurements can be analyzed to determine sets of biomarkers (i.e., their levels) that best discriminate between the different classifications of the training set via an optimization procedure. The analysis of protein expression data can include training a machine learning model to distinguish between positive and negative samples based on the levels of certain protein biomarkers. The analysis can include using the data as a training set where the biomarker levels and known diagnosis are used to train a machine learning model to distinguish between positive and negative samples. In the process of learning, the model identifies protein biomarkers that are predictive for BCL-2 inhibitor response.

In some embodiments, the measuring comprises the detection and quantification (e.g., semi-quantification) of the selected biomarkers in the sample. In some embodiments, the measured biomarker levels are adjusted relative to one or more standard level(s) (“normalized”). As known in the art, normalizing is done to remove technical variability inherent to a platform to give a quantity or relative quantity (e.g., of lipid classes or species, or metabolites).

In some embodiments, the measurement of differential levels of specific biomarkers from biological samples may be accomplished using a range of technologies, reagents, and methods. These include any of the methods of measurement as described elsewhere herein.

The biomarker levels are typically normalized following detection and quantification as appropriate for the particular platform using methods routinely practiced by those of ordinary skill in the art.

In particular embodiments, protein expression signatures may be obtained using hierarchical clustering and predictive models of protein classifiers, e.g., as determined by classification and regression trees (CART) analysis. In some embodiments, a supervised statistical approach known as sparse linear classification is used in which sets of gene products are identified by the model according to their ability to separate phenotypes during a training process that uses the selected set of patient samples. The outcomes of training is a biomarker signature(s) and classification coefficients for the classification comparison. Together the signature(s) and coefficient(s) provide a classifier or predictor. Training may also be used to establish threshold or cut-off values. Threshold or cut-off values can be adjusted to change test performance, e.g., test sensitivity and specificity. For example, the threshold for HCC may be intentionally lowered to increase the sensitivity of the test for HCC, if desired.

Determining the accuracy of classification may involve the use of accuracy measures such as sensitivity, specificity, positive predictive value (PPV), negative predictive value (NPV), accuracy, and area under the curve (AUC) of a Receiver Operating Characteristic (ROC) curve corresponding to the diagnostic accuracy of detecting or predicting HCC.

In some embodiments, the classifiers that are developed during training and using a training set of samples are applied for prediction purposes to diagnose new individuals (“classification”). For each subject or patient, a biological sample is taken and the normalized biomarker levels (i.e., the relative amounts of biomarkers) in the sample of each of the biomarkers specified by the signatures found during training are the input for the classifier. In other embodiments, the classifier can also use the weighting coefficients discovered during training for each gene product. As outputs, the classifiers are used to compute probability values. Each probability value may be used to determine the likelihood of responding to a BCL-2 inhibitor in the subject.

In some embodiments, these values may be reported relative to a reference range that indicates the confidence with which the classification is made. In some embodiments, the output of the classifier may be compared to a threshold value, for example, to report a “positive” in the case that the classifier score or probability exceeds the threshold indicating a high likelihood of responding to a BCL-2 inhibitor. If the classifier score or probability fails to reach the threshold, the result would be reported as “negative” for the respective condition.

It should be noted that a classifier obtained with one platform may not show optimal performance on another platform. This could be due to the promiscuity of probes or other technical issues particular to the platform. Accordingly, also described herein are methods to adapt a signature as taught herein from one platform for another.

It will be appreciated that for any particular biomarker, a distribution of biomarker levels for AML patients responding or not responding to a BCL-2 inhibitor may overlap. Under such conditions, a test does not absolutely distinguish the two populations (i.e., responders vs non-responders) with 100% accuracy, and the area of overlap indicates where the test cannot distinguish them. A threshold value is selected, above which the test is considered to be “positive” and below which the test is considered to be “negative.” In some embodiments, the values for one or more of the biomarkers listed in Table 1 are dichotomized into yes/no fits in a cluster 1 or 2 profile, as described in the Examples, and the overall cluster designation is designated based on the sum of these findings.

In some embodiments, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more biomarkers are selected to discriminate between subjects that respond or do not respond to a BCL-2 inhibitor with at least about 70%, 75%, 80%, 85%, 90%, 95% accuracy or having a C-statistic of at least about 0.70, 0.75, 0.80, 0.85, 0.90, 0.95.

In some embodiments, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more biomarkers are selected to discriminate between subjects that respond favorably, respond unfavorably, or respond very unfavorably to a BCL-2 inhibitor with at least about 70%, 75%, 80%, 85%, 90%, 95% accuracy or having a C-statistic of at least about 0.70, 0.75, 0.80, 0.85, 0.90, 0.95.

In some embodiments, a subject is determined to have a significant probability of having or not having a specified condition or outcome (e.g., responding to a BCL-2 inhibitor). By “significant probability” is meant that the subject has a reasonable probability (0.6, 0.7, 0.8, 0.9 or more) of having, or not having, a specified condition or outcome.

In some embodiments, a determination of a BCL-2 responder status in an AML patient can be based not solely on biomarker levels, but can also take into account clinical and/or other data about the subject, e.g., clinical data about the subject's current medical state, the medical history of the subject, demographic data about the subject (age, sex, etc.), or the results of one or more laboratory tests.

### E. Treatment Decisions

The determination of a likelihood of responding to a BCL-2 inhibitor in an AML patient using the present methods and compositions can be used to inform the delivery of medical care appropriate for the AML in the subject. For example, a determination that a subject has a high likelihood of responding to a BCL-2 inhibitor can lead to a decision to continue or initiate a BCL-2 inhibitor therapy in a subject. Alternatively, a determination that a subject has a low likelihood of responding to a BCL-2 inhibitor can lead to a decision to discontinue and/or not initiate a BCL-2 inhibitor therapy, and/or to initiate an alternative therapy for the patient.

Thus, in one aspect, provided herein is a method for treating AML in a subject comprising, consisting essentially of, or consisting of: administering an effective amount of an BCL-2 inhibitor-comprising AML treatment to a subject having differential levels of one or more AML response biomarkers in a biological sample from the subject as compared to a control, wherein the one or more biomarkers comprise one or more of SPI1, NOTCH.cle, PTPN12, and/or any one or more of the proteins listed in Table 1, in particular a protein listed in Table 1 that is underlined or in bold. In some instances, the subject has differential levels of one or more of SPI1, NOTCH.cle, PTPN12, wherein (i) the relative expression level of SPI1 is determined to be greater than or equal to −2, and the relative expression level of NOTCH1.cle is determined to be greater than 2.2, or (ii) the relative expression level of SPI1 is determined to be less than −2, and the relative expression level of PTPN12 is determined to be less than 1.2.

Thus, in another aspect, provided herein is a method for treating AML in a subject comprising, consisting essentially of, or consisting of: administering an effective amount of an BCL-2 inhibitor-comprising AML treatment to a subject having differential levels of one or more AML therapy response biomarkers in a biological sample from the subject as compared to a control, wherein the one or more biomarkers comprise one or more of the proteins listed in Table 3, Table 4, and/or Table 9. In some instances, the subject has differential levels of one or more of CDKN1B.pT198, MSH6, ASH2L, MDM2.pS166, or ITGA2.

In another aspect, provided herein is a method of treating AML in a subject, the method comprising administering to the subject a therapeutically effective amount of a BCL-2 inhibitor, wherein the subject has been identified as a likely responder to the BCL-2 inhibitor based on a detection of the protein levels of one or more AML therapy response biomarkers in a biological sample comprising AML cells obtained from the subject, and wherein the identification of the subject as a likely responder is based on a difference in the one or more protein levels relative to protein levels of the one or more AML therapy response biomarkers in a biological sample from a healthy individual without AML. In particular embodiments, the one or more biomarkers comprise one or more of SPI1, NOTCH.cle, PTPN12, and/or any one or more of the proteins listed in Table 1, particular protein in Table 1 that is underlined or in bold. In some instances, the subject has differential levels one or more of SPI1, NOTCH.cle, PTPN12, wherein (i) the relative expression level of SPI1 is determined to be greater than or equal to −2 (i.e., greater than 2 log base 2 below control), and the relative expression level of NOTCH1.cle is determined to be greater than 2.2 (i.e., greater than 2.2 log base 2 above control), or (ii) the relative expression level of SPI1 is determined to be less than −2, and the relative expression level of PTPN12 is determined to be less than 1.2.

In particular embodiments, the one or more biomarkers comprise one or more of the proteins listed in Table 3, Table 4, and/or Table 9. In some instances, the subject has differential levels one or more of one or more of CDKN1B.pT198, MSH6, ASH2L, MDM2.pS166, or ITGA2.

In some embodiments, the method comprises: providing a biological sample comprising AML cells from a subject with AML; detecting the one or more AML response biomarkers in the sample, and comparing the levels of the one or more biomarkers to a control, wherein the, the one or more biomarkers comprise one or more of SPI1, NOTCH.cle, PTPN12, and/or any one or more of the proteins listed in Table 1, particular protein in Table 1 that is underlined or in bold. In some embodiments, the method comprises determining a BCL-2 inhibitor response status in the subject according to the decision tree shown in FIG. 2. In some instances, the subject has differential levels one or more of SPI1, NOTCH.cle, PTPN12, wherein (i) the relative expression level of SPI1 is determined to be greater than or equal to −2, and the relative expression level of NOTCH1.cle is determined to be greater than 2.2, or (ii) the relative expression level of SPI1 is determined to be less than −2, and the relative expression level of PTPN12 is determined to be less than 1.2.

In some embodiments, the method comprises: providing a biological sample comprising AML cells from a subject with AML; detecting the one or more AML response biomarkers in the sample and comparing the levels of the one or more biomarkers to a control, wherein the one or more biomarkers comprise one or more of the proteins listed in Table 3, Table 4, or Table 9. In some embodiments, the method comprises determining a BCL-2 inhibitor response status. In some instances, the subject has differential levels one or more of CDKN1B.pT198, MSH6, ASH2L, MDM2.pS166, or ITGA2.

The subject with AML (e.g., a subject determined to be a responder) can be treated with a BCL-2 inhibitor. The BCL-2 inhibitor can be venetoclax, BCL201, navitoclax, oblimersen, gossypol compounds, obatoclax, and/or others. In particular embodiments, the BCL-2 inhibitor is venetoclax. Any BCL-2 inhibitor can be used in the herein-disclosed methods, including venetoclax, BCL201, navitoclax, oblimersen, gossypol compounds, obatoclax, and others. In some embodiments, the BCL-2 inhibitor is venetoclax. Venetoclax is a BH3-mimetic that is a highly selective inhibitor of BCL-2. Venetoclax acts intracellularly in a BCL2-overexpressing leukemic cell to initiate apoptosis by mimicking the action of the endogenous antagonists of BCL2, the BH3-only proteins. Heightened expression of BCL2 protects leukemia cells from apoptosis by inhibiting activation of BAX and BAK, even when normally lethal cellular stresses induce pro-death BH3-only proteins such as BIM and NOXA. Venetoclax interacts with BCL-2 selectively in the BH3-binding groove to directly and indirectly (via release of BIM) relieve repression of BAX/BAK, which dimerize to permeabilize mitochondria and unleash apoptosis through release of cytochrome C and subsequent caspase activation. See Roberts, A. W., Hematology Am Soc Hematol Educ Program, 2020 Dec. 4; 2020(1):1-9. doi: 10.1182/hematology.2020000154.

In some embodiments, the subject is treated with one or more additional or alternative therapies for AML (i.e., in addition to an administration of a BCL-2 inhibitor, or in place of an administration of a BCL-2 inhibitor in subjects determined to have a low likelihood of responding to a BCL-2 inhibitor). Such additional or alternative therapies can include, e.g., conventional chemotherapy. Any “conventional chemotherapy” can be used in the herein-disclosed methods. “Conventional chemotherapy” comprises chemotherapeutic treatments that are widely accepted and used by most health professionals. In some embodiments, the CC is a chemotherapeutic agent such as cytosine arabinoside (trade name cytarabine (ara-C, or A), including low, standard or high-dose cytosine arabinoside (A), typically in combination with other agents such as cladribine (C), fludarabine (F), an anthracycline (e.g., daunorubicin (D), idarubicin (I), doxorubicin, or mitoxantrone (M)) and a growth factor (e.g., granulocyte colony-stimulating factor (G-CSF)) (G), to give combinations such as IA, CIA, FIA, FIAG, FLAG-I, among others. Such additional or alternative therapies can also or alternatively include, e.g., targeted therapies (e.g., FLT3 inhibitors (e.g., first generation multi-kinase inhibitors such as sorafenib, lestaurtinib, midostaurin and next generation inhibitors such as quizartinib, crenolanib, gilteritinib), isocitrate dehydrogenase (IDH) inhibitors (e.g., enasidenib, ivosidenib), monoclonal antibodies (e.g., gemtuzumab ozogamicin; see also Williams, B. A., et al. 2019, J. Clin. Med. 8(8):1261; doi: 10.3390/jcm8081261), hedgehog pathway inhibitors (e.g., vismodegib, sonidegib, arsenic trioxide; see also Jamieson, C. et al. 2022, Blood Cancer Discov. 1(2):134-145, DOI: 10.1158/2643-3230.BCD-20-0007), non-chemotherapy drugs such as all-trans retinoic acid or arsenic trioxide, surgery, radiation, stem cell transplants (e.g., allogeneic hematopoietic stem-cell transplants), and others (e.g., menin inhibitors, CD47 antibodies, TP53 targeting agents).

In some embodiments, a patient receiving a BCL-2 inhibitor therapy (e.g., an identified responder) or not receiving a BCL-2 inhibitor therapy (e.g., an identified non-responder) is treated with one or more additional or alternative therapies. For example, in some embodiments, the subject is treated with chemotherapy, a targeted therapy (e.g., FLT3 inhibitors, IDH inhibitors, monoclonal antibodies such as gemtuzumab oxogamicin, hedgehog pathway inhibitors), a non-chemotherapy drug such as all-trans retinoic acid or arsenic trioxide, surgery, radiation, stem cell transplants, and others. In particular embodiments, a subject receiving a BCL-2 inhibitor is also treated with conventional chemotherapy, e.g., a chemotherapeutic agent such as cytarabine (ara-C), particularly low-dose cytarabine, and/or a hypomethylating agent (HMA) such as decitabine or azacitidine.

### F. Kits and Systems

### 1. Kits

In one aspect, kits are provided for the determination of the likelihood of a subject with AML responding to a BCL-2 inhibitor such as venetoclax, wherein the kits can be used to detect the biomarkers described herein. The kit may include, e.g., one or more agents for the detection of biomarkers, a container for holding a biological sample, e.g., blood sample or bone marrow sample, isolated from a human subject with AML; and instructions for reacting agents with the biological sample or a portion of the biological sample to detect the presence or amount of at least one biomarker in the biological sample. The agents may be packaged in separate containers. The kit may further comprise one or more control reference samples and reagents for performing the herein-described methods. The kit may also comprise one or more devices or implements for carrying out any of the herein methods.

The provided kits can contain reagents for detecting one or more biomarkers as described above in Section C. In certain embodiments, the kit comprises agents for measuring the levels of one or more of SPI1, NOTCH.cle, or PTPN12, e.g., antibodies specific to SPI1, NOTCH.cle, or PTPN12 proteins. In some embodiments, the kit comprises agents for measuring the levels of one or more of SPI1, NOTCH.cle, or PTPN12, e.g., agents for performing an ELISA assay. In some embodiments, the kit comprises agents for measuring the protein levels of any one or more biomarkers or sets of biomarkers listed in Table 1, and particularly a protein listed in Table 1 that is underlined and/or bold.

In some embodiments, the kit comprises agents for measuring the protein levels of any one or more biomarkers or sets of biomarkers listed in Table 3, Table 4, or Table 9. In certain embodiments, the kit comprises agents for measuring the levels of one or more of CDKN1B.pT198, MSH6, ASH2L, MDM2.pS166, or ITGA2, e.g., antibodies specific to CDKN1B.pT198, MSH6, ASH2L, MDM2.pS166, or ITGA2 proteins. In some embodiments, the kit comprises agents for measuring the levels of one or more of CDKN1B.pT198, MSH6, ASH2L, MDM2.pS166, or ITGA2, e.g., agents for performing an ELISA assay

The kit can comprise one or more containers for compositions contained in the kit. Compositions can be in liquid form or can be lyophilized. Suitable containers for the compositions include, for example, bottles, vials, syringes, and test tubes. Containers can be formed from a variety of materials, including glass or plastic. The kit can also comprise a package insert containing instructions for methods of determining the BCL-2 inhibitor response status of a subject with AML as described above in Section D.

In some embodiments, kit design will involve prospective validation for the protein expression values obtained from the protein measurement method used in the kit to obtain the appropriate cluster classification, including new random forests based on the biomarker protein expression measured via both RPPA (as described in the Examples) and the selected protein detection method for the kit (e.g., ELISA) that will most accurately learn the transduction function (“cutoff values”) necessary to convert the protein expression values measured using the selected expression detection method to the appropriate cluster classification, as discussed above in Section C.

### 2. Measurement Systems and Reports for Detecting and Recording Biomarker Expression

In one aspect, a system, e.g., measurement system is provided. Such systems allow, e.g., the detection of biomarker levels in a sample and the recording of the data resulting from the detection. The stored data can then be analyzed to determine the BCL-2 inhibitor response status of a subject such as described above in Section D. Such systems can comprise, e.g., assay systems (e.g., comprising an assay device and detector), which can transmit data to a logic system (such as a computer or other system or device for capturing, transforming, analyzing, or otherwise processing data from the detector). The logic system can have any one or more of multiple functions, including controlling elements of the overall system such as the assay system, sending data or other information to a storage device or external memory, and/or issuing commands to a treatment device.

Also provided is a system for detecting AML response biomarkers in a sample, by utilizing a station for analyzing the sample by ELISA assay or RPPA, wherein the biomarkers comprise one or more of SPI1, NOTCH.cle, PTPN12, and/or any of the biomarkers listed in Table 1, particularly biomarkers in Table 1 that are underlined or in bold; the sample is a biological sample, e.g., blood sample or bone marrow sample, obtained from a subject with AML, and the report is useful for determining the likelihood that the subject will respond to a therapy comprising a BCL-2 inhibitor such as venetoclax. Optionally, a station for generating a report containing information on results of the analyzing is further included.

Also provided is a system for detecting AML response biomarkers in a sample, by utilizing a station for analyzing the sample by ELISA assay or RPPA, wherein the biomarkers comprise one or more of any of the biomarkers listed in Table 3, Table 4, or Table 9; such as for example one or more of CDKN1B.pT198, MSH6, ASH2L, MDM2.pS166, or ITGA2, and wherein the sample is a biological sample, e.g., blood sample or bone marrow sample, obtained from a subject with AML, and the report is useful for determining the likelihood that the subject will respond to a therapy comprising a BCL-2 inhibitor such as venetoclax. Optionally, a station for generating a report containing information on results of the analyzing is further included.

Also provided is a method of generating a report containing information on results of the detection of AML response biomarkers in a sample, including detecting one or more AML response biomarkers in the sample, and generating the report, wherein the one or more biomarkers comprise one or more of any of the biomarkers listed in Table 1, particularly biomarkers in Table 1 underlined or in bold, such as for example one or more of SPI1, NOTCH.cle, PTPN12; and wherein the sample is a biological sample, e.g., blood sample or bone marrow sample, obtained from a subject with AML, and the report is useful for determining the likelihood that the subject will respond to a therapy comprising a BCL-2 inhibitor such as venetoclax.

Also provided is a method of generating a report containing information on results of the detection of AML response biomarkers in a sample, including detecting one or more AML response biomarkers in the sample, and generating the report, wherein the one or more biomarkers comprise one or more of any of the biomarkers listed in Table 3, Table 4, or Table 9, such as for example one or more of CDKN1B.pT198, MSH6, ASH2L, MDM2.pS166, or ITGA2; and wherein the sample is a biological sample, e.g., blood sample or bone marrow sample, obtained from a subject with AML, and the report is useful for determining the likelihood that the subject will respond to a therapy comprising a BCL-2 inhibitor such as venetoclax.

### 3. Computer Diagnostic Systems for Determining BCL-2 Inhibitor Responder Status

Certain aspects of the herein-described methods may be totally or partially performed with a computer system including one or more processors, which can be configured to perform the steps. Thus, embodiments are directed to computer systems configured to perform the steps of methods described herein, potentially with different components performing a respective step or a respective group of steps. The computer systems of the present disclosure can be part of a measuring system as described above, or can be independent of any measuring systems. In some embodiments, the present disclosure provides a computer system that uses inputted biomarker expression (and optionally other) data, and determines the BCL-2 inhibitor responder status of a subject.

A computer system can include desktop and laptop computers, tablets, mobile phones and other mobile devices. The system can include various elements such as a printer, keyboard, storage device(s), monitor (e.g., a display screen, such as an LED), peripherals, devices to connect a computer system to a wide area network such as the Internet, a mouse input device, scanner, a storage device(s), computer readable medium, camera, microphone, accelerometer, and the like. Any of the data mentioned herein can be output from one component to another component and can be output to the user.

In one aspect, the present disclosure provides a computer implemented method for determining the likelihood of responding to a BCL-2 inhibitor in a patient with AML. The computer performs steps comprising, for example: receiving inputted patient data comprising values for the levels of one or more biomarkers in a biological sample from the patient (e.g., determined as described above in Section C); analyzing the levels of one or more biomarkers and optionally comparing them to respective reference values, optionally comparing the biomarker levels to one or more threshold values to determine BCL-2 inhibitor responder status (e.g., determined as described above in Section D); and displaying information regarding the responder status or probability in the patient. In certain embodiments, the inputted patient data comprises values for the levels of a plurality of biomarkers in a biological sample from the patient, e.g., one or more of any of the biomarkers listed in Table 1, particularly biomarkers in Table 1 underlined or in bold, such as for example one or more of SPI1, NOTCH.cle, PTPN12. In certain embodiments, the inputted patient data comprises values for the levels of a plurality of biomarkers in a biological sample from the patient, e.g., one or more of any of the biomarkers listed in Table 3, Table 4, or Table 9, such as for example one or more of CDKN1B.pT198, MSH6, ASH2L, MDM2.pS166, or ITGA2.

In a further aspect, a diagnostic system is included for performing the computer implemented method, as described. A diagnostic system may include a computer containing a processor, a storage component (i.e., memory), a display component, and other components typically present in general purpose computers. The storage component stores information accessible by the processor, including instructions that may be executed by the processor and data that may be retrieved, manipulated or stored by the processor.

The storage component includes instructions for determining the BCL-2 inhibitor responder status of the subject. For example, the storage component includes instructions for determining responder status based on biomarker levels, as described herein. The computer processor is coupled to the storage component and configured to execute the instructions stored in the storage component in order to receive patient data and analyze patient data according to one or more algorithms. The display component displays information regarding the diagnosis of the patient. The storage component may be of any type capable of storing information accessible by the processor, such as a hard-drive, memory card, ROM, RAM, DVD, CD-ROM, USB Flash drive, write-capable, and read-only memories.

The instructions may be any set of instructions to be executed directly (such as machine code) or indirectly (such as scripts) by the processor. In that regard, the terms “instructions,” “steps” and “programs” may be used interchangeably herein. The instructions may be stored in object code form for direct processing by the processor, or in any other computer language including scripts or collections of independent source code modules that are interpreted on demand or compiled in advance.

Data may be retrieved, stored or modified by the processor in accordance with the instructions. For instance, although the diagnostic system is not limited by any particular data structure, the data may be stored in computer registers, in a relational database as a table having a plurality of different fields and records, XML documents, or flat files. The data may also be formatted in any computer-readable format such as, but not limited to, binary values, ASCII or Unicode. Moreover, the data may comprise any information sufficient to identify the relevant information, such as numbers, descriptive text, proprietary codes, pointers, references to data stored in other memories (including other network locations) or information which is used by a function to calculate the relevant data. In certain embodiments, the processor and storage component may comprise multiple processors and storage components that may or may not be stored within the same physical housing. For example, some of the instructions and data may be stored on removable CD-ROM and others within a read-only computer chip. Some or all of the instructions and data may be stored in a location physically remote from, yet still accessible by, the processor. Similarly, the processor may actually comprise a collection of processors which may or may not operate in parallel. In one aspect, computer is a server communicating with one or more client computers. Each client computer may be configured similarly to the server, with a processor, storage component and instructions. Although the client computers and may comprise a full-sized personal computer, many aspects of the system and method are particularly advantageous when used in connection with mobile devices capable of wirelessly exchanging data with a server over a network such as the Internet.

### Exemplary Embodiments

In one aspect, the present disclosure provides a method of determining the likelihood that a subject with acute myeloid leukemia (AML) will respond to a therapy comprising the administration of a BCL-2 inhibitor, the method comprising detecting protein levels of one or more AML therapy response biomarkers in a biological sample comprising AML cells obtained from the subject, wherein the one or more AML therapy response biomarkers comprises a biomarker listed in Table 3, Table 4, Table 6, Table 7, or Table 8. In some embodiments, the AML therapy response biomarkers comprises one or more biomarkers listed in Table 9. In some embodiments, 2, 3, 4, 5, 6, 7, 8, 9, 10, or more AML therapy response biomarkers are detected in the biological sample. In some embodiments, the AML therapy response biomarkers comprise one or more of CDKN1B.pT198, MSH6, ASH2L, MDM2.pS166, or ITGA2. In some embodiments, the AML therapy response biomarkers comprise one or more of PRMT1, SPI1, or EIF2AK2. In some embodiments, the AML therapy response biomarkers comprise one or more of CASP9, KMT2D, or SMARCB1. In some embodiments, the AML therapy response biomarkers comprise one or more of NOTCH1.cle, PRMT1, and CHEK2.

In some embodiments, the method further comprises calculating a response score based on the detected protein levels of the AML therapy response biomarkers, wherein the response score corresponds to the likelihood that the subject will respond to the therapy. In some embodiments, the subject is determined to have a high likelihood of responding to the therapy. In some embodiments, the method further comprises administering the therapy to the subject. In some embodiments, the subject is determined to have a low likelihood of responding to the therapy. In some embodiments, the method further comprises administering an alternative therapy to the subject that does not comprise the BCL-2 inhibitor. In some embodiments, the BCL-2 inhibitor is venetoclax. In some embodiments, a response to the therapy comprises an increase in remission duration. In some embodiments, a response to the therapy comprises an increase in overall survival. In some embodiments, the biological sample is a blood sample or bone marrow sample. In some embodiments, the protein levels of the one or more AML therapy response biomarkers are measured using one or more antibodies that specifically bind to the one or more biomarkers.

In another aspect, the present disclosure provides a method of generating a report containing information on the likelihood that a subject with AML will respond to a therapy comprising a BCL-2 inhibitor, comprising: detecting in a biological sample obtained from the subject the protein levels of one or more AML therapy response biomarkers; and, generating the report, wherein the one or more AML therapy response biomarkers comprise one or more biomarkers listed in Table 3, Table 4, Table 6, Table 7, or Table 8, and wherein the report is useful for determining the likelihood that the subject will respond to the therapy. In some embodiments, the one or more AML therapy response biomarkers comprise one or more of the biomarkers listed in Table 9.

In some embodiments, the one or more AML therapy response biomarkers comprise at least one of CDKN1B.pT198, MSH6, ASH2L, MDM2.pS166, or ITGA2. In some embodiments, the AML therapy response biomarkers comprise one or more of PRMT1, SPI1, or EIF2AK2. In some embodiments, the AML therapy response biomarkers comprise one or more of CASP9, KMT2D, or SMARCB1. In some embodiments, the AML therapy response biomarkers comprise one or more of NOTCH1.cle, PRMT1, and CHEK2.

In another aspect, the present disclosure provides a system for determining the likelihood that a subject with AML will respond to a therapy comprising a BCL-2 inhibitor, comprising a station for analyzing a biological sample comprising AML cells obtained from the subject to measure protein levels of one or more AML therapy response biomarkers in the sample, wherein the one or more AML therapy response biomarkers comprise one or more biomarkers listed in Table 3, Table 4, Table 6, Table 7, or Table 8.

In some embodiments, the AML therapy response biomarkers comprise at least one of the biomarkers listed in Table 9. In some embodiments, the one or more AML therapy response biomarkers comprise at least one of CDKN1B.pT198, MSH6, ASH2L, MDM2.pS166, or ITGA2. In some embodiments, the AML therapy response biomarkers comprise one or more of PRMT1, SPI1, or EIF2AK2. In some embodiments, the AML therapy response biomarkers comprise one or more of CASP9, KMT2D, or SMARCB1. In some embodiments, the AML therapy response biomarkers comprise one or more of NOTCH1.cle, PRMT1, and CHEK2. In some embodiments, the system further comprises a station for generating a report containing information on results of the analyzing.

In another aspect, the present disclosure provides a method of treating a subject with acute myeloid leukemia (AML), the method comprising administering to the subject a therapeutically effective amount of a BCL-2 inhibitor to a subject having differential levels of one or more AML response biomarkers in a biological sample from the subject as compared to a control, wherein the one or more biomarkers comprise at least one of CDKN1B.pT198, MSH6, ASH2L, MDM2.pS166, or ITGA2, and/or any one or more of biomarkers listed in Table 3, Table 4, Table 6, Table 7, Table 8, or Table 9. In some embodiments, the AML therapy response biomarkers comprise one or more of PRMT1, SPI1, or EIF2AK2. In some embodiments, the AML therapy response biomarkers comprise one or more of CASP9, KMT2D, or SMARCB1. In some embodiments, the AML therapy response biomarkers comprise one or more of NOTCH1.cle, PRMT1, and CHEK2.

In another aspect, the present disclosure provides a method of treating a subject with acute myeloid leukemia (AML), the method comprising administering to the subject a therapeutically effective amount of a BCL-2 inhibitor, wherein the subject has been identified as a likely responder to the BCL-2 inhibitor based on a detection of the protein levels of one or more AML therapy response biomarkers in a biological sample comprising AML cells obtained from the subject, wherein the identification of the subject as a likely responder is based on a difference in the one or more protein levels relative to protein levels of the one or more AML therapy response biomarkers in a biological sample from a healthy individual without AML, and wherein the one or more AML therapy response biomarkers comprises a biomarker listed in Table 3, Table 4, Table 6, Table 7, or Table 8. In some embodiments, the AML therapy response biomarkers comprise at least one of the biomarkers listed in Table 9. In some embodiments, 2, 3, 4, 5, 6, 7, 8, 9, 10, or more AML therapy response biomarkers are detected in the biological sample. In some embodiments, the AML therapy response biomarkers comprise at least one of CDKN1B.pT198, MSH6, ASH2L, MDM2.pS166, or ITGA2. In some embodiments, the AML therapy response biomarkers comprise one or more of PRMT1, SPI1, or EIF2AK2. In some embodiments, the AML therapy response biomarkers comprise one or more of CASP9, KMT2D, or SMARCB1. In some embodiments, the AML therapy response biomarkers comprise one or more of NOTCH1.cle, PRMT1, and CHEK2.

In some embodiments, the identification of the subject as a likely responder comprises the calculation of a response score based on the detected protein levels of the AML therapy response biomarkers, wherein the response score corresponds to the likelihood that the subject will respond to the therapy. In some embodiments, the BCL-2 inhibitor is venetoclax. In some embodiments, the administration of the therapy leads to an increase in remission duration. In some embodiments, the administration of the therapy leads to an increase in overall survival. In some embodiments, the biological sample is a blood sample or bone marrow sample. In some embodiments, wherein the protein levels of the one or more AML therapy response biomarkers are measured using one or more antibodies that specifically bind to the one or more biomarkers. In some embodiments, the protein levels are measured using an ELISA assay.

In another aspect, the present disclosure comprises a method of generating a report containing information on the likelihood that a subject with AML will not respond to a therapy comprising a BCL-2 inhibitor, comprising detecting in a biological sample obtained from the subject the protein levels of one or more AML therapy response biomarkers; and, generating the report, wherein the one or more AML therapy response biomarkers comprise one or more biomarkers listed in Table 3, Table 4, Table 6, Table 7, Table 8, or Table 9, and wherein the report is useful for determining the likelihood that the subject will not respond to the therapy. In some embodiments, the AML therapy response biomarkers comprise one or more biomarkers listed in Table 9. In some embodiments, the one or more AML therapy response biomarkers comprise at least one of CDKN1B.pT198, MSH6, ASH2L, MDM2.pS166, or ITGA2. In some embodiments, the AML therapy response biomarkers comprise one or more of PRMT1, SPI1, or EIF2AK2. In some embodiments, the AML therapy response biomarkers comprise one or more of CASP9, KMT2D, or SMARCB1. In some embodiments, the AML therapy response biomarkers comprise one or more of NOTCH1.cle, PRMT1, and CHEK2.

In one aspect, the present disclosure provides a method of determining the likelihood that a subject with acute myeloid leukemia (AML) will respond to a therapy comprising the administration of a BCL-2 inhibitor, the method comprising detecting protein levels of one or more AML therapy response biomarkers in a biological sample comprising AML cells obtained from the subject, wherein the one or more AML therapy response biomarkers comprises a biomarker listed in Table 1.

In some embodiments, the method further comprises calculating a response score based on the detected protein levels of the AML therapy response biomarkers, wherein the response score corresponds to the likelihood that the subject will respond to the therapy. In some embodiments, the subject is determined to have a high likelihood of responding to the therapy. In some embodiments, the method further comprises administering the therapy to the subject. In some embodiments, the subject is determined to have a low likelihood of responding to the therapy. In some embodiments, the method further comprises administering an alternative therapy to the subject that does not comprise the BCL-2 inhibitor. In some embodiments, the BCL-2 inhibitor is venetoclax. In some embodiments, a response to the therapy comprises an increase in remission duration. In some embodiments, a response to the therapy comprises an increase in overall survival. In some embodiments, the biomarker listed in Table 1 is correlated with remission duration (bolded) and/or overall survival (underlined).

In some embodiments, the AML therapy response biomarkers comprise SPI1, NOTCH.cle, and PTPN12. In some embodiments, the relative expression level of the SPI1 biomarker as compared to control is greater than or equal to −2, and the relative expression level of the NOTCH.cle biomarker as compared to control is less than 2.2; or the relative expression level of the SPI1 biomarker as compared to control is less than −2 and the relative expression level of the PTPN12 biomarker as compared to control is greater than or equal to 1.2. In some embodiments, the subject is determined to have a low likelihood of responding to the therapy. In some embodiments, the relative expression level of the SPI1 biomarker as compared to control is greater than or equal to −2 and the relative expression level of the NOTCH.cle marker is greater than or equal to 2.2; or the relative expression level of the SPI1 biomarker as compared to control is less than −2 and the relative expression level of the PTPN12 biomarker as compared to control is less than 1.2. In some embodiments, the subject is determined to have a high likelihood of responding to the therapy.

In some embodiments, the therapy comprises Ara-C and/or a hypomethylating agent (HMA). In some embodiments, 2, 3, 4, 5, 6, 7, 8, 9, 10, or more AML therapy response biomarkers are detected in the biological sample. In some embodiments, the biological sample is a blood sample or bone marrow sample. In some embodiments, the protein levels of the one or more AML therapy response biomarkers are measured using one or more antibodies that specifically bind to the one or more biomarkers.

In another aspect, the present disclosure provides a method of generating a report containing information on the likelihood that a subject with AML will respond to a therapy comprising a BCL-2 inhibitor, comprising: detecting in a biological sample obtained from the subject the protein levels of one or more AML therapy response biomarkers; and, generating the report, wherein the one or more AML therapy response biomarkers comprise one or more biomarkers listed in Table 1, and wherein the report is useful for determining the likelihood that the subject will respond to the therapy.

In some embodiments, the one or more AML therapy response biomarkers comprise SPI1, NOTCH.cle, and PTPN12.

In another aspect, the present disclosure provides a system for determining the likelihood that a subject with AML will respond to a therapy comprising a BCL-2 inhibitor, comprising a station for analyzing a biological sample comprising AML cells obtained from the subject to measure protein levels of one or more AML therapy response biomarkers in the sample, wherein the one or more AML therapy response biomarkers comprise one or more biomarkers listed in Table 1.

In some embodiments, the one or more AML therapy response biomarkers comprise SPI1, NOTCH.cle, and PTPN12. In some embodiments, the system further comprises a station for generating a report containing information on results of the analyzing.

In another aspect, the present disclosure provides a method of treating a subject with acute myeloid leukemia (AML), the method comprising administering to the subject a therapeutically effective amount of a BCL-2 inhibitor to a subject having differential levels of one or more AML response biomarkers in a biological sample from the subject as compared to a control, wherein the one or more biomarkers comprise one or more of SPI1, NOTCH.cle, PTPN12, and/or any one or more of the proteins listed in Table 1.

In another aspect, the present disclosure provides a method of treating a subject with acute myeloid leukemia (AML), the method comprising administering to the subject a therapeutically effective amount of a BCL-2 inhibitor, wherein the subject has been identified as a likely responder to the BCL-2 inhibitor based on a detection of the protein levels of one or more AML therapy response biomarkers in a biological sample comprising AML cells obtained from the subject, wherein the identification of the subject as a likely responder is based on a difference in the one or more protein levels relative to protein levels of the one or more AML therapy response biomarkers in a biological sample from a healthy individual without AML, and wherein the one or more AML therapy response biomarkers comprises a biomarker listed in Table 1.

In some embodiments, the identification of the subject as a likely responder comprises the calculation of a response score based on the detected protein levels of the AML therapy response biomarkers, wherein the response score corresponds to the likelihood that the subject will respond to the therapy. In some embodiments, the BCL-2 inhibitor is venetoclax. In some embodiments, the administration of the therapy leads to an increase in remission duration. In some embodiments, the administration of the therapy leads to an increase in overall survival. In some embodiments, the biomarker listed in Table 1 is correlated with remission duration (bolded) and/or overall survival (underlined). In some embodiments, the AML therapy response biomarkers comprise SPI1, NOTCH.cle, and PTPN12.

In some embodiments, the relative expression level of the SPI1 biomarker as compared to control is greater than or equal to −2 and the relative expression level of the NOTCH.cle marker is greater than or equal to 2.2; or the relative expression level of the SPI1 biomarker as compared to control is less than −2 and the relative expression level of the PTPN12 biomarker as compared to control is less than 1.2. In some embodiments, the therapy comprises also administering a therapeutic amount of Ara-C and/or a hypomethylating agent (HMA) to the subject. In some embodiments, 2, 3, 4, 5, 6, 7, 8, 9, 10, or more AML therapy response biomarkers are detected in the biological sample. In some embodiments, the biological sample is a blood sample or bone marrow sample. In some embodiments, the protein levels of the one or more AML therapy response biomarkers are measured using one or more antibodies that specifically bind to the one or more biomarkers. In some embodiments, the protein levels are measured using an ELISA assay.

### EXAMPLES

### Example 1. Preliminary Proteomic Signature Prognostic for Response to Venetoclax in Adults with Acute Myeloid Leukemia

Reverse Phase Protein Array (RPPA) as described by Tibes et al. 2006 and Kornblau et al. 2011 was performed on leukemia blast enriched fractions prepared from diagnostic samples of 818 adults with AML, of which 143 were treated with venetoclax (VTX) including 33 in combination with high dose Ara-C, 5 with standard dose Ara-C, 50 with hypomethylating agents (HMA), and 13 with HMA and targeted therapy (either CPX-351 liposome, a dual-drug liposomal encapsulation of cytarabine and daunorubicin, or anthracycline). Protein expression levels were evaluated using 390 validated antibodies against total and post translationally modified (phosphorylated, methylated, cleaved) as described by Hoff et al. 2019. proteins and were analyzed in the context of clinical data compiled by retrospective chart review. Pearson correlation was used to identify significant protein-protein correlations. Survival curves were generated by the Kaplan-Meier method and survival data was analyzed by multivariate cox regression model. Protein expression signatures were identified by hierarchical clustering and predictive models of protein classifiers were determined by classification and regression trees (CART) analysis.

We queried the 390 proteins (Table 1) assayed in the 143 VTX treated patients to identify proteins individually prognostic for overall survival (OS) or remission duration (REMDUR) and identified 27 proteins correlated with OS (p<0.01) and 44 proteins correlated with REMDUR (p<0.01). Neither MCL1, BCLXL nor BCL2 expression at diagnosis were prognostic of overall survival (OS) or remission duration (REMDUR). Unbiased hierarchical clustering of the 44-protein classifier correlated with REMDUR demonstrated two cohorts (n=102 and n=41) of patients by protein expression signatures.

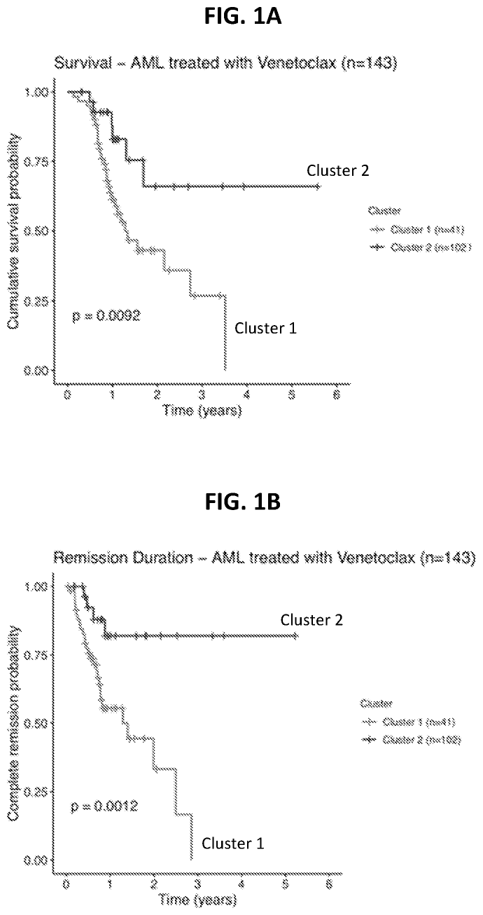

Survival analysis showed significant differences between clusters with estimated 3-yr overall survival 27% in cluster 1 compared to 66% in cluster 2 (p=0.009, FIG. 1A) and relapse risk (RR) at 1-yr 45% versus 18% (p=0.001, FIG. 1B), respectively. Remission rates were insignificantly less in cluster 1 (61% vs 77%). The clusters were similar for clinical features (Table 2) with no significant differences noted for age, gender, performance status, cytogenetics risk classification, or the presence of molecular mutation markers FLT3.ITD, IDH1/2, NPM1, or TP53.

In multivariate analysis, protein cluster membership was an independent prognostic factor for OS along with TP53 and NPM1 mutations, but unfavorable cytogenetics was not. Prognostication did not vary based on cytogenetics or the other therapy agent used in combination with VTX. For REMDUR, protein cluster membership and unfavorable cytogenetics were the only independent predictors.

CART modeling of the VTX treated cohort identified 3 proteins (FIG. 2)—SPI1, NOTCH1.cle, and PTPN12—that predicted cluster membership with a computed accuracy of 94.3% (misclassification error 6%). Similarly, when these three proteins were used as training variables for random forest classification, the error rate was 3.7%.

Protein expression patterns, individually and in combination, were very highly predictive of outcome to VTX containing combination chemotherapy. A group with lower response rates, higher relapse rates, shorter REMDUR and inferior OS was defined. A diagnostic kit, likely using readily available technology such as ELISA, with the three classifier proteins could be generated and used to identify patients likely to remain in remission versus relapse with VTX based therapies. This could be used to select patients that have received VTX containing regimens for continued therapy, for those unlikely to relapse, or alternate/further therapy in remission for those likely to relapse. Additionally, many previously unrecognized potential therapeutic targets for potentially preventing or overcoming VTX resistance were identified amongst the other 44 proteins associated with REMDUR. Further testing in the laboratory is needed to investigate these interactions and explore potential clinical relevance. If targeting of these proteins in combination with VTX can lead to improved outcomes, use of inhibitors of proteins with elevated expression would be applicable clinically in combination with VTX to improve clinical outcomes.

TABLE 1
Evaluated proteins (n = 390) correlated (p < 0.01) with remission duration (bold) and overall survival (underlined) in adult
patients with AML treated with venetoclax (n = 143).

| | | | | | | | | | |
|---|---|---|---|---|---|---|---|---|---|
| ABL1 | BID | CLPP | ERG | HES1 | LRP6.pS1490 | NOTCH3 | PRKCA.pS657 | RSKA1_2_3 | SUZ12 |
| ABL1.pY412 | BIRC2 | COPS5 | ERN1 | HEXIM1 | LYN | NPM1 | PRKCB | S100A4 | SYK |
| ACACA | BIRC5 | CREB1 | ETS1 | HIF1A | MAP1LC3A_B | NRAS | PRKCB.pS660 | SCD | TAPBP |
| ACACA.pS79 | BMI1 | CREB1.pS133 | EZH2 | HIST3H3 | MAP2K1 | NUMB | PRMT1 | SDHA | TFRC |
| ADAR | BRAF | CSK | EZR | HK2 | MAP2K1.pS217_221 | NUP98 | PRMT5 | SETD1A | TGM2 |
| AIFM1 | BRAF.pS445 | CSNK2A1 | FASN | HNRNPK | MAPK1 | OMA1 | PRMT7 | SETD1B | TIGAR |
| AKR1C3 | BRD4 | CTNNB1.pS33_37_T41 | FLI1 | HSF1 | MAPK1_3.pT202_Y204 | P90RSK.pT573 | PTEN | SF3B1 | TNFRSF4 |
| AKT1 | BTK | CTNNB1.pT41_S45 | FN1 | HSF1.pS326 | MAPK14 | PAK1 | PTGS2 | SF3B1.pT313 | TNK1 |
| AKT1_2_3 | CASP3 | CXCR5 | FOS | HSP60 | MAPK14.pT180 Y182 | PAK4 | PTK2 | SGK1 | TP53 |
| AKT1_2_3.pS473 | CASP3.cle | DAPK1.pS308 | FOXM1 | HSP90AA1_B1 | MAPK7 | PARP | PTK2.pY397 | SGK3 | TP53BP1 |
| AKT1_2_3.pT308 | CASP7.cle | DDB1 | FOXO3 | HSPB1 | MCL1 | PARP.cle214 | PTPN11 | SHC1.pY317 | TRAP1 |
| AKT1S1 | CASP9 | DLST | FOXO3.pS318_ | HSPB1.pS82 | MDM2 | PAX6 | PTPN11.pY542 | SIRT1 | TRIM28 |
| | | | 321 | | | | | | |
| AKT1S1.pT246 | CAV1 | DLX1 | FRS2A.pY196 | IDO1 | MDM2.pS166 | PCNA | PTPN12 | SIRT6 | TSC1 |
| AKT2 | CBL | DNM1L | FZR1 | IGF1R | MECOM | PDCD1 | PTPN6 | SMAD1 | TSC2 |
| AKT3 | CBX7 | DNMT1 | G6PD | IGF1R.pY1135_1136 | MEF2C | PDCD4 | PTPN6.pY564 | SMAD2 | TSC2.pT1462 |
| AMBRA1.pS52 | CCNB1 | DUSP4 | GAB2 | IGFBP2 | MEN1 | PDGFRB | PXN | SMAD2.pS245_ | TUBA1A |
| | | | | | | | | 250_255 | |
| ANXA1 | CCND1 | DUSP6 | GAB2.pY452 | INPP4B | MET.pY1230_ | PDHK1 | PYK2.pY402 | SMAD2.pS465_ | TYRO3 |
| | | | | | 1234_1235 | | | 467 | |
| ANXA7 | CCND3 | DVL3 | GAPDH | IRS1 | MKNK1 | PDK1 | RAB11 | SMAD5.pS463_ | UGT1A1 |
| | | | | | | | | 465 | |
| ARAF | CD276 | E2F1 | GATA1 | IRS2 | MSH2 | PDK1.pS241 | RAD50 | SNAI1 | ULK1.pS757 |
| ARID1A | CD4 | EEF2 | GATA3 | ITGA2 | MSH6 | PDL1 | RAD51 | SOD1 | VASP |
| ASH2L | CD44 | EEF2K | GLS | ITGAL | MSI2 | PEA15 | RAF1 | SOD2 | VAV1 |
| ASNS | CD70 | EGFR | GRP75 | ITGB1 | MSLN | PECAM1 | RAF1.pS338 | SOX17 | VCP |
| ASS1 | CD74 | EIF2AK2 | GRP78 | JAG1 | MTOR | PHLPP | RB1 | SOX2 | VDAC1 |
| ATF3 | CD86 | EIF2AK3 | GSKA_B | JAK2 | MTOR.pS2448 | PIK3CA | RB1.pS807_ | SPARC | VHL |
| | | | | | | | 811 | | |
| ATG3 | CDC25C | EIF2S1 | GSKA_B.pS21_9 | JMJD6 | MUC1 | PIK3CB | RELA_P100 | SPI1 | VIM |
| ATG4B | CDH1 | EIF2S1.pS51 | GYS1 | JUN.pS73 | MYC | PIK3R1 | RELA_P65 | SPP1 | VTCN1 |
| ATG7 | CDK1 | EIF4E | GYS1.pS641 | JUNB | MYH11 | PIM2 | RELA_P65.pS536 | SRC | WDR5 |
| AURORA_A_ | CDK1_2_3.pT14 | EIF4E.pS209 | H2AX.pS139 | KAT2A | MYH9.pS1943 | PKM2 | RHEB | SRC.pY527 | WDR77 |
| B_C.pT288_232_198 | | | | | | | | | |
| AXL | CDK2 | EIF4EBP1 | H2BFM | KDM1A | NCL | PLCG1.pS1248 | RICTOR.pT1135 | SRSF1 | WDR82 |
| BABAM1.pS29 | CDKN1A | EIF4EBP1.pS65 | H3K27Me3 | KDR | NCSTN | PLCG2.pY759 | RIP3 | SSBP2 | WEE1 |
| BAK1 | CDKN1B | EIF4G1 | H3K36Me3 | KEAP1 | NDRG1.pT346 | PLK1 | RIPK1 | STAT1 | WEE1.pS642 |
| BAX | CDKN1B.pS10 | EIF4G2 | H3K4Me1 | KIT | NDUFB4 | PPARA | RPA32 | STAT1.pY701 | WIPI2 |
| BBC3 | CDKN1B.pT198 | ELK1.pS383 | H3K4Me2 | KMT2A | NF2 | PPP2R2A_B_C_D | RPA32.pS4_8 | STAT3 | WTAP |
| BCL2 | CDKN2A | EP300 | H3K4Me3 | KMT2D | NFE2L2 | PREX1 | RPS6 | STAT3.pS727 | XPA |
| BCL2.pS70 | CDX2 | EPHA2 | H3K9Me2 | LATS1 | NLN | PRKAA1_2 | RPS6.pS235_236 | STAT3.pY705 | XPF |
| BCL2A1 | CHEK1 | EPHA2.pS897 | HDAC1 | LCK | NOL3 | PRKAA1_2.pT172 | RPS6.pS240_244 | STAT5A | XRCC1 |
| BCL2L1 | CHEK1.pS345 | EPHA2.pY588 | HDAC2 | LEF1 | NOTCH1 | PRKAA2.pS345 | RPS6KB1 | STK11 | YAP1.pS127 |
| BCL2L11 | CHEK2 | ERBB2 | HDAC3 | LGALS3 | NOTCH1.cle | PRKAR1A | RPS6KB1.pT389 | STK4 | YWHAE |
| BECN1 | CHEK2.pT68 | ERBB2.pY1248 | HDAC6 | LMNB1 | NOTCH2 | PRKCA | RPTOR | STMN1 | ZAP70 |

TABLE 2
Clinical characteristics of patients in two cohorts by hierarchical clustering
of 44-protein classifier correlated with remission duration.

| | | Cluster 1 | Cluster 2 | p |
|---|---|---|---|---|
| | | N (%)* | N (%)* | value |
| Gender | Male | 55 (53.9) | 23 (56.1) | 0.960 |
| | Female | 47 (46.1) | 18 (43.9) | |
| Age (years) | Mean (SD) | 64.5 (14.0) | 64.5 (13.8) | 0.998 |
| Age Group | 18-40 y.o. | 10 (9.8) | 3 (7.3) | 0.794 |
| | 41-60 y.o. | 16 (15.7) | 8 (19.5) | |
| | >60 y.o. | 76 (74.5) | 30 (73.2) | |
| Race | Asian | 1 (1.0) | 2 (4.9) | 0.264 |
| | Black/AA | 3 (3.0) | 4 (9.8) | |
| | Hispanic | 4 (4.0) | 1 (2.4) | |
| | White/Caucasian | 87 (87.9) | 32 (78.0) | |
| | Other/Unknown | 4 (4.0) | 2 (4.9) | |
| Performance | Asymptomatic | 15 (16.3) | 7 (17.5) | 0.942 |
| Status | Symptomatic | 52 (56.5) | 24 (60.0) | |
| | Bed < 50% | 23 (25.0) | 8 (20.0) | |
| | Bed > 50% | 2 (2.2) | 1 (2.5) | |
| Prior | No | 68 (67.3) | 31 (77.5) | 0.324 |
| Malignancy | Yes | 33 (32.7) | 9 (22.5) | |
| WBC | Mean (SD) | 21.2 (37.2) | 12.8 (43.7) | 0.261 |
| (cells/μl) | | | | |
| Hyper- | No | 93 (93.0) | 37 (97.4) | 0.567 |
| leukocytosis | Yes | 7 (7.0) | 1 (2.6) | |
| PLT | Mean (SD) | 83.1 (132.1) | 60.1 (38.7) | 0.293 |
| (cells/μl) | | | | |
| Best Response | CR/CRi | 44 (71.0) | 26 (86.7) | 0.192 |
| | HI/PR/SD/NR | 9 (14.5) | 3 (10.0) | |
| | Death/ED | 9 (14.5) | 1 (3.3) | |
| Remission | No | 34 (34.3) | 8 (20.5) | 0.192 |
| | Yes | 60 (60.6) | 30 (76.9) | |
| | ED | 5 (5.1) | 1 (2.6) | |
| Remission | Mean (s.d.) | 41.3 (29.8) | 64.7 (59.5) | 0.015 |
| Duration | | | | |
| Relapse | No | 33 (55.0) | 26 (86.7) | 0.006 |
| AraC | No | 54 (52.9) | 27 (65.9) | 0.222 |
| | Yes | 48 (47.1) | 14 (34.1) | |
| AraC Dose | Missing | 54 (52.9) | 27 (65.9) | 0.556 |
| | Low | 19 (18.6) | 5 (12.2) | |
| | Standard | 4 (3.9) | 1 (2.4) | |
| | High | 25 (24.5) | 8 (19.5) | |
| CPX-351 | No | 98 (96.1) | 40 (97.6) | 1.000 |
| | Yes | 4 (3.9) | 1 (2.4) | |
| Anthracycline | No | 74 (72.5) | 32 (78.0) | 0.640 |
| | Yes | 28 (27.5) | 9 (22.0) | |
| HMA | No | 44 (43.1) | 14 (34.1) | 0.423 |
| | Yes | 58 (56.9) | 27 (65.9) | |
| Cytogenetics | Favorable | 1 (1.0) | 0 (0.0) | 0.073 |
| Risk Group | Intermediate | 51 (51.5) | 27 (73.0) | |
| | Unfavorable | 47 (47.5) | 10 (27.0) | |
| FLT3.ITD | No | 64 (90.1) | 19 (86.4) | 0.916 |
| mutated | Yes | 7 (9.9) | 3 (13.6) | |
| FLT3.ITD | No | 5 (83.3) | 1 (33.3) | 0.453 |
| high allelic ratio | Yes | 1 (16.7) | 2 (66.7) | |
| IDH | No | 57 (74.0) | 14 (63.6) | 0.493 |
| mutated | Yes | 20 (26.0) | 8 (36.4) | |
| MLL | No | 4 (100.0) | 5 (83.3) | 1.000 |
| mutated | Yes | 0 (0.0) | 1 (16.7) | |
| NPM1 | No | 60 (82.2) | 18 (75.0) | 0.636 |
| mutated | Yes | 13 (17.8) | 6 (25.0) | |
| TP53 | No | 49 (65.3) | 17 (63.0) | 1.000 |
| mutated | Yes | 26 (34.7) | 10 (37.0) | |

Legend:
Bed, time spent in bed;
WBC, white blood cell count;
HMA, hypomethylating agents;
PLT, platelet count;
CR, complete remission;
CRi, complete remission with incomplete count recovery;
HI, hematologic improvement;
PR; partial response;
SD, stable disease;
NR, no response;
ED, early death.

### Example 2. Proteomic Signatures Prognostic for Response, Relapse, and Survival in Adults with Acute Myeloid Leukemia

The initial data as described in Example 1 was based on an analysis of 390 antibodies with clinical follow up of the patients through April 2021. Subsequently, we added an additional 22 antibodies, for a total of 412, into the set, and obtained updated follow-up information on the patients through August 2022. The enlarged dataset, with longer follow-up, was then subjected to the same analysis as before, and an updated set of biomarkers was identified having improved discriminatory capability, as described in this Example. As presented below, three prognostic groups are recognized: good, intermediate, and bad. The list of proteins that were significant for outcome increased from 27 to 36. Likewise, the algorithms to identify the optimal proteins for the classifier sets, now recognizing three groups instead of two, were also redone and superior predictive capability was achieved. This improved analysis is presented below.

### A. Methods

Sample Collection and Processing. Peripheral blood (PB) and bone marrow (BM) samples were collected from 818 adult patients with newly diagnosed AML, of which 143 received VTX-containing induction chemotherapy, evaluated at the University of Texas MD Anderson Cancer Center (MDACC) between January 2012 and July 2020. Informed consent was obtained in accordance with the Declaration of Helsinki. Samples were processed and analyzed under an Investigational Review Board (IRB) approved laboratory protocol (Lab 06-0565).

The following methods for sample collection and processing, protein lysate and array slide printing were performed for all 818 adult patient samples as well as ten cryopreserved normal bone marrow-derived CD34+ samples from healthy subjects (normal control). Whole cell AML blast lysates were generated from fresh peripheral blood (n=273 overall, n=46 VTX cohort) and bone marrow (n=545 overall, n=97 VTX cohort) patient samples. Leukemic cells were enriched by Ficoll separation to isolate a mononuclear cell fraction followed by CD3/19 magnetic T and B cell depletion. To prepare lysates, the cell concentration was normalized to a concentration of 10,000 cells μL-1 and 10 million leukemia blast-enriched cells were suspended in 500 μL PBS, lysed in 500 μL of boiling hot protein lysis buffer (Tris buffered saline pH 7.4, 10% SDS, 2% beta-mercaptoethanol).

Reverse Phase Protein Array. Proteomic profiling of the patient samples was performed by Reverse Phase Protein Array (RPPA) methodology as previously described in full. [Ref. 7-9] Briefly, cell lysates were diluted in five serial dilutions in 96-well plates and transferred into 384-well plates. Plates were loaded into the Aushon 2470 arrayer and lysate material was printed onto nitrocellulose-coated glass slides with a single touch per dot. The five serial dilutions gave printed dots with approximately 85, 42, 21, 11, and 5 cell equivalents of protein, respectively.

Proteins of interest were analyzed using strictly-validated primary antibodies to probe the slides. Slides were probed with 412 strictly-validated primary antibodies including 324 against total proteins (Table 3—Standard Font), 78 against phosphorylation sites (Table 3—Bold), 4 targeting cleavage forms (Table 3—Bold Italics), and 6 histone methylation sites (Table 3—Bold Underline) followed by a secondary antibody for signal amplification. Stained slides were scanned using InnoScan 710 InfraRed microarray scanner and analyzed using Microvigene softeware to produce quantitative protein expression data. We used the nomenclature system previously described whereby post-translation modifications are denoted by a period that follows the protein name, then the type of post-translation modification, ‘p’ for phosphorylation, ‘cle’ for cleaved, and ‘Me’ for methylation, followed by the letter abbreviation for the affected amino acid and finally its sequence position. [Ref 10]

TABLE 3
412 Proteins analyzed in the RPPA Methodologies.

| | | | | |
|---|---|---|---|---|
| ABL1 | BCL2L11 | CHEK1.pS345 | EPHA2 | H3K4Me3 |
| ABL1.pY412 | BCL11A | CHEK2 | EPHA2.pS897 | H3K9Me2 |
| ACACA | BECN1 | CHEK2.pT68 | EPHA2.pY588 | HDAC1 |
| ACACA.pS79 | BID | CLPP | ERBB2 | HDAC2 |
| ADAR | BIRC2 | COPS5 | ERBB2.pY1248 | HDAC3 |
| ADM | BIRC5 | CREB1 | ERG | HDAC6 |
| AIFM1 | BMI1 | CREB1.pS133 | ERN1 | HES1 |
| AKR1C3 | BRAF | CRM1 | ESR1 | HEXIM1 |
| AKT1 | BRAF.pS445 | CSE1L | ETS1 | HIF1A |
| AKT1_2_3 | BRD4 | CSK | EZH2 | HIST3H3 |
| AKT1_2_3.pS473 | BTK | CSNK2A1 | EZR | HK2 |
| AKT1_2_3.pT308 | CASP3 | CTCF | FASN | HNRNPK |
| AKTIS1 | CASP3.cle | CTNNB1.pS33 | FLI1 | HSF1 |
| AKT1S1.pT246 | CASP7.cle | CTNNB1.pT41 | FN1 | HSF1.pS326 |
| AKT2 | CASP9 | CXCR5 | FOS | HSP60 |
| AKT3 | CAV1 | DAPK1.pS308 | FOXM1 | HSP90AA1_B1 |
| AMBRA1.pS52 | CBL | DDB1 | FOXO3 | HSPB1 |
| ANXA1 | CBX7 | DLST | FOXO3.pS318 | HSPB1.pS82 |
| ANXA7 | CCNB1 | DLX1 | FRS2A.pY196 | IDO1 |
| AR | CCND1 | DNM1L | FZR1 | IGF1R |
| ARAF | CCND3 | DNMT1 | G6PD | IGF1R.pY1135 |
| ARID1A | CD276 | DPF2 | GAB2 | IGFBP2 |
| ASH2L | CD4 | DUSP4 | GAB2.pY452 | IKB |
| ASNS | CD44 | DUSP6 | GAPDH | IL23R |
| ASS1 | CD70 | DVL3 | GATA1 | INPP4B |
| ATF3 | CD74 | E2F1 | GATA3 | IRS1 |
| ATG3 | CD86 | EEF2 | GLS | IRS2 |
| ATG4B | CDC25C | EEF2K | GPX4 | ITGA2 |
| ATG7 | CDH1 | EGFR | GRP75 | ITGAL |
| AURKA | CDK1 | EIF2AK2 | GRP78 | ITGB1 |
| AURORA.pT288 | CDK1_2_3.pT14 | EIF2AK3 | GSKA_B | JAG1 |
| AXL | CDK2 | EIF2S1 | GSKA_B.pS21 | JAK2 |
| BABAM1.pS29 | CDK9 | EIF2S1.pS51 | GYS1 | JMJD6 |
| BAK1 | CDKN1A | EIF4E | GYS1.pS641 | JUN.pS73 |
| BAP1 | CDKN1B | EIF4E.pS209 | H2AX.pS139 | JUNB |
| BAX | CDKN1B.pS10 | EIF4EBP1 | H2BFM | KAT2A |
| BBC3 | CDKN1B.pT198 | EIF4EBP1.pS65 | H3K27Ac | KDM1A |
| BCL2 | CDKN2A | EIF4G1 | H3K27Me3 | DR |
| BCL2.pS70 | CDT1 | EIF4G2 | H3K36Me3 | KEAP1 |
| BCL2A1 | CDX2 | ELK1.pS383 | H3K4Me1 | KIT |
| BCL2L1 | CHEK1 | EP300 | H3K4Me2 | KMT2A |
| KMT2D | NLN | PRKAA2.pS345 | RPTOR | STK11 |
| LATS1 | NOL3 | PRKAR1A | RSKA1_2_3 | STK4 |
| LATS2 | NOTCH1 | PRKCA | S100A4 | STMN1 |
| LCK | NOTCH1.cle | PRKCA.pS657 | SCD | SUZ12 |
| LEF1 | NOTCH2 | PRKCB | SDHA | SYK |
| LGALS3 | NOTCH3 | PRKCB.pS660 | SETD1A | TAPBP |
| LMNB1 | NPM1 | PRMT1 | SETD1B | TFRC |
| LRP6.pS1490 | NRAS | PRMT5 | SF3B1 | TGM2 |
| LYN | NUMB | PRMT7 | SF3B1.pT313 | TIGAR |
| MAPILC3A B | NUP98 | PTEN | SFRP1 | TNFRSF4 |
| MAP2K1 | OMA1 | PTGS2 | SGK1 | TNK1 |
| MAP2K1.pS217 | P90RSK.pT573 | PTK2 | SGK3 | TP53 |
| MAPK1 | PAK1 | PTK2.pY397 | SHC1.pY317 | TP53BP1 |
| MAPK1_3.pT202 | PAK4 | PTPN11 | SIRT1 | TRAP1 |
| MAPK14 | PARP | PTPN11.pY542 | SIRT6 | TRIM28 |
| MAPK14.pT180 | PARP.cle214 | PTPN12 | SMAD1 | TSC1 |
| MAPK7 | PAX6 | PTPN6 | SMAD2 | TSC2 |
| MCL1 | PCNA | PTPN6.pY564 | SMAD2.pS245 | TSC2.pT1462 |
| MDM2 | PDCD1 | PXN | SMAD2.pS465 | TUBAIA |
| MDM2.pS166 | PDCD4 | PYK2.pY402 | SMAD5.pS463 | TXNRD1 |
| MECOM | PDGFRB | RAB11 | SMARCA2 | TYRO3 |
| MEF2C | PDHK1 | RAD50 | SMARCA4 | UGT1A1 |
| MEN1 | PDK1 | RAD51 | SMARCB1 | ULK1.pS757 |
| MET.pY1230 | PDK1.pS241 | RAF1 | SNAI1 | VASP |
| MKNK1 | PDL1 | RAF1.pS338 | SOD1 | VAV1 |
| MSH2 | PEA15 | RB1 | SOD2 | VCP |
| MSH6 | PECAM1 | RB1.pS807 | SOX17 | VDAC1_VDAC3 |
| MSI2 | PHLPP | RELA_P100 | SOX2 | VHL |
| MSLN | PIK3CA | RELA_P65 | SPARC | VIM |
| MTOR | PIK3CB | RELA_P65.pS536 | SPI1 | VTCN1 |
| MTOR.pS2448 | PIK3R1 | RHEB | SPP1 | WDR5 |
| MUC1 | PIM2 | RICTOR.pT1135 | SRC | WDR77 |
| MYC | PKM2 | RIP3 | SRC.pY527 | WDR82 |
| MYH11 | PLCG1.pS1248 | RIPK1 | SRSF1 | WEE1 |
| MYH9.pS1943 | PLCG2.pY759 | RPA32 | SSBP2 | WEE1.pS642 |
| NCL | PLK1 | RPA32.pS4_8 | STAT1 | WIPI2 |
| NCSTN | PPARA | RPS6 | STAT1.pY701 | WTAP |
| NDRG1.pT346 | PPP2R2A_B_C_D | RPS6.pS235 | STAT3 | XPA |
| NDUFB4 | PREX1 | RPS6.pS240 | STAT3.pS727 | XPF |
| NF2 | PRKAA1_2 | RPS6KB1 | STAT3.pY705 | XRCC1 |
| NFE2L2 | PRKAA1_2.pT172 | RPS6KB1.pT389 | STAT5A | YAP1.pS127 |
| YWHAE | | | | |
| ZAP70 | | | | |

Proteins (324);
Phosphorylated Proteins (78);
Cleaved Proteins (4);
Methylated Proteins (6)

Proteomic Analysis. Initial computational quality control steps were performed as previously described to ensure proper slide alignment, background noise control, and sample loading control. [Ref 10] The Supercurve algorithm was used to generate a single expression value from the five serial dilutions. [Ref 11] Protein expression levels in AML patient samples were median-normalized for each protein relative to the median expression of 10 bone marrow derived CD34+ samples from healthy subjects (control samples) also printed on the array. For each protein, expression was median-normalized with the median of the normal control samples set to zero. Positive final expression values indicate increased expression relative to the controls, while negative final expression values indicate decreased expression relative to the controls.

Following data normalization, a cohort of 143 patients that received VTX-containing induction chemotherapy was identified for separate analysis for the purposes of this manuscript. Survival curves were generated by Kaplan-Meier method and survival data was analyzed by multivariate cox regression model. [Refs. 12, 13] Protein expression signatures were identified by hierarchical Progeny clustering. [Ref. 14] Correlations between clustered protein signatures and clinical features were assessed using Fisher's exact test for categorical variables and the Kruskal-Wallis test for continuous variables. Multivariate analysis for survival outcomes OS and RD was performed by Cox regression model. Median protein expression within clusters was compared to median expression in the normal controls to identify significant expression trends with the Wilcoxon test. Protein networks were built using Cytoscape (version 3.9.1) combining literature-based protein interactions queried from the STRING database and proteomic-based interactions inferred from the RPPA data derived from Pearson correlation. [Ref. 11] Predictive classifier models were assembled in Python using the random forest machine learning technique. All bioinformatics and analysis were performed in Rstudio (version 1.3.1093) with R (version 4.1.2).

Protein expression validation in AML cell lines. MOLM-13 parental and VTX-resistant cells were the kind gift of Dr. Marina Konopleva (MD Anderson Cancer Center; Houston, TX, USA). OCI-AML-3, KG-1, and MV4-11 cells were obtained from the Leukemia Sample Bank (LSB) at MD Anderson. For protein expression comparison in cell lines, immunoblot analysis was performed. Cells were boiled and sonicated in lysis buffer and protein (1.5×10⁵ cell equivalents) was subjected to electrophoresis using SDS/PAGE. Immunoblot analysis was performed with antibodies against CBL (BD Transduction Lab), PRKCB.pS660 (Cell Signaling, Beverly, MA), SYK (Santa Cruz, Dallas, TX), PKM2 (Cell Signaling), STAT3.pS727 (Cell Signaling), Beta-Actin (Sigma Aldrich, St. Louis, MO), GADPH (Ambion, Austin, TX) and Alpha-Tubulin (Sigma Aldrich). Signals were detected by using the ProteinSimple FluorChem imaging system and quantified by AlphaView software. Beta-Actin, GAPDH, and Alpha-Tubulin were used as loading controls. Densitometry was calculated as protein expression/expression of housekeeping gene, relative to the control.

### B. Results

### 1. Patient Characteristics

A cohort of 143 patients that received VTX as a component of induction chemotherapy was identified, of which 27 received VTX combined with high-dose cytarabine (Ara-C), 29 combined with low- or standard-dose Ara-C, 80 with hypomethylating agents, 6 with high-dose Ara-C and a hypomethylating agent, and 12 combined with targeted therapy (5 FLT3 inhibitors, 5 IDH inhibitors, 2 TP53 inhibitors). In the VTX cohort, 5 patients were not treated at MDACC leaving 138 patients evaluable for outcome and further analysis. Median age at diagnosis was 68 years (range 18-90 years) with median follow-up 58 weeks (range 2-396 weeks). Remission (CR/CRi) was achieved in 65% of patients (n=90) of which 40% relapsed (n=36). Patient demographic, diagnostic labs, (data not shown) treatment, and outcome data was compiled to be analyzed against protein expression data.

### 2. Patient Clustering by Protein Expression

For each protein, expression across samples were divided by medians, tertiles (low, middle, and high expression), sextiles, high ⅓ vs low ⅔, high ⅔ vs low ⅓ and survival curves were generated by Kaplan-Meier (log rank) method for OS and remission duration (RD) by expression category. Thirty-six proteins were identified with expression correlated (Bonferroni corrected p<0.01) to OS (Table 4) and 44 proteins correlated to RD. Kaplan-Meier plots for STAT3.pS727 (p<0.0001) and MAPK14.pT180 (p<0.0001) for OS and RD, were generated (data not shown).

TABLE 4
36 proteins individually correlated to OS (p < 0.01) ranked by significance.

| PROTEIN NAME | P VALUE | PROTEIN NAME | P VALUE |
|---|---|---|---|
| HSF1.pS326 | 0.0001 | ERG | 0.0024 |
| SCD | 0.0001 | CHEK2 | 0.0025 |
| STAT3.pS727 | 0.0001 | AKR1C3 | 0.0030 |
| CDKN1B.pS10 | 0.0002 | MSH6 | 0.0032 |
| COPS5 | 0.0002 | EIF2AK2 | 0.0037 |
| KMT2D | 0.0002 | VTCN1 | 0.0037 |
| MAPK14.pT180_Y182 | 0.0005 | PRMT1 | 0.0038 |
| CDKN1B | 0.0006 | RAF1.pS338 | 0.0038 |
| CD74 | 0.0010 | RB1 | 0.0055 |
| ASH2L | 0.0010 | BCL2A1 | 0.0059 |
| STAT3.pY705 | 0.0010 | PTPN12 | 0.0062 |
| YWHAE | 0.0014 | SGK1 | 0.0065 |
| PEA15 | 0.0017 | TSC2 | 0.0070 |
| WIPI2 | 0.0019 | TNFRSF4 | 0.0079 |
| TNK1 | 0.0020 | CASP7.cle | 0.0080 |
| WDR5 | 0.0020 | JAG1 | 0.0084 |
| AR | 0.0023 | EIF4EBP1.pS65 | 0.0085 |
| EP300 | 0.0023 | ATF3 | 0.0097 |

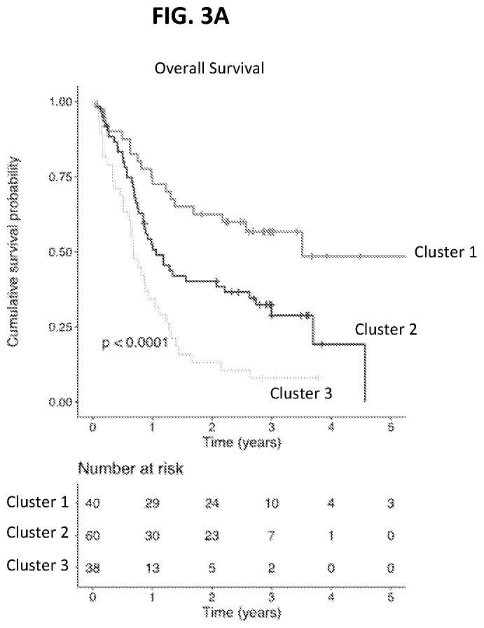

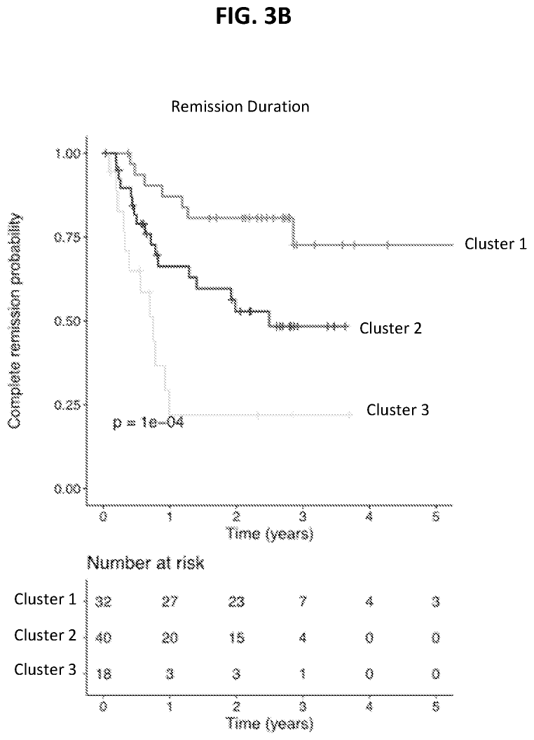

Next, recurring patterns of expression were identified across the 36 proteins correlated to OS (FIGS. 3A and 3B) by applying the Progeny clustering algorithm utilizing k-means, and unbiased hierarchical clustering [Ref. 14] identifying three distinct protein expression patterns Cluster 1 (C1; n=41), Cluster 2 (C2; n=63), and Cluster 3 (C3; n=39). Protein expression was somewhat heterogeneous between clusters for several proteins, namely AR, STAT3.pY705, and RAF1.pS338. Expression of CDKN1B and CDKN1B.pS10 were nearly universally high in AML samples relative to the CD34+ controls, albeit phosphorylated CDKN1B was relatively higher in C1 members. While universally low across AMVL samples, ASH2L and MSH6 were relatively lower in the C1 signature as were PRMT1, MAPK14.pT180, HSF1.pS326, EIF4EBP1.pS65, EIF2AK2, and WDR5. The C3 signature was marked by relatively high expression of PrMT1, KMT2D, STAT3.pS727, EP300, and TSC2 and relatively low expression in AKR1C3, YWHAE, TNK1, COPS5, ATF3, SGK1, VTCN1, TNFRSF4, and PEA15.

3. Protein Signature Correlates with Response, Relapse and Survival.

The clinical outcomes of patients were significantly different across clusters with C3 and C2 having inferior survival outcomes compared to C1 with C3 being particularly poor. Remission rates were significantly less in C3 compared to C2 and C1 (50% vs 70% and 82%; p=0.029). Similarly, in the 90 patients that achieved remission (C1 n=32, C2 n=40, C3 n=18), relapse rates were significantly higher in C3 (66.7%) compared to C2 and C1 (42.5% and 21.9%; p=0.02) with shorter median remission duration (39 vs 129 weeks and C1 median not reached; p<0.001). Survival was markedly shorter in C3 with median OS 35.7 weeks compared to 55.1 and 182.7 weeks (p<0001) in C2 and C1, respectively. When the same 36-protein classifier and Progeny clustering algorithm was applied to the 667 patients that did not receive VTX, similar clustering of the proteins was observed (data not shown); but there were no differences in survival or relapse free survival between the clusters (data not shown), indicating the protein classifier to be specific for the response to VTX-containing therapy.

Comparison of the three clusters showed no differences for basic demographics, molecular mutation risk stratifiers, or for the induction chemotherapy regimen combined with VTX. This included no differences between the clusters for gender, age, race, prior malignancy, primary or secondary disease status, induction chemotherapy classes, or historical molecular risk classifiers including TP53, IDH1/2, NPM1, EZH2, SRSF2, and FLT3 (data not shown). The groups differed by established cytogenetic risk stratifiers (p=0.021) with C3 having a relatively higher percentage of unfavorable (66.7%; Table 5). Importantly, however, C1 contained 29.7% unfavorable risk cytogenetics despite superior survival outcomes and C3 the only patient with favorable risk cytogenetics. The majority of patients in C1 and C2 were intermediate risk cytogenetics (70.3% and 60%). There were also significant differences between the three clusters noted for BM blast percentage as well as PB total white blood cell count (WBC), blast percentage and absolute blast count.(Table 5).

TABLE 5
Clinical characteristics with significant differences noted between groups.

| | Overall | Cluster 1 | Cluster 2 | Cluster 3 | P- |
|---|---|---|---|---|---|
| | (N = 143) | (N = 41) | (N = 63) | (N = 39) | value* |
| Achieved | | | | | 0.029 |
| Remission | | | | | |
| N = 132 (ED | | | | | |
| Removed) | | | | | |
| No | 42 (31.8%) | 7 (17.9%) | 17 (29.8%) | 18 (50.0%) | |
| Yes | 90 (68.2%) | 32 (82.1%) | 40 (70.2%) | 18 (50.0%) | |
| Relapse | | | | | 0.02 |
| No | 54 (60.0%) | 25 (78.1%) | 23 (57.5%) | 6 (33.3%) | |
| Yes | 36 (40.0%) | 7 (21.9%) | 17 (42.5%) | 12 (66.7%) | |
| Remission | | | | | <0.001 |
| Duration (Weeks) | | | | | |
| Median [95 CI] | | NA | 129 [67, NA] | 39 [20, NA] | |
| Survival (Weeks) | | | | | <0.001 |
| Median [95 CI] | | 182.7 [87.9, NA] | 55.1 [43.8, | 35.7 [26.6, 57.7] | |
| | | | 136.8] | | |
| BM Blast | | | | | <0.001 |
| Percentage (at | | | | | |
| Diagnosis) | | | | | |
| Mean (SD) | 46.2 (24.3) | 35.7 (20.2) | 45.0 (23.8) | 58.7 (23.8) | |
| Median [Min, | 41.0 [1.00, 95.0] | 28.0 [1.00, 77.0] | 40.0 [1.80, 95.0] | 65.5 [2.00, 91.0] | |
| Max] | | | | | |
| WBC (at | | | | | 0.003 |
| Diagnosis) | | | | | |
| Mean (SD) | 18.8 (39.1) | 6.52 (12.1) | 23.9 (49.9) | 23.0 (35.1) | |
| Median [Min, | 3.55 [0.500, 265] | 2.00 [0.600, | 3.50 [0.600, 265] | 8.20 [0.500, 144] | |
| Max] | | 56.6] | | | |
| PB Blast | | | | | <0.001 |
| Percentage (at | | | | | |
| Diagnosis) | | | | | |
| Mean (SD) | 24.6 (27.6) | 10.2 (15.7) | 23.4 (27.3) | 40.5 (29.2) | |
| Median [Min, | 13.0 [0, 95.0] | 4.00 [0, 61.0] | 14.0 [0, 95.0] | 40.0 [0, 95.0] | |
| Max] | | | | | |
| PB Absolute Blast | | | | | <0.001 |
| Count | | | | | |
| (at Diagnosis) | | | | | |
| Mean (SD) | 10100 (29300) | 2210 (6540) | 13300 (38400) | 12900 (25300) | |
| Median [Min, | 388 [0, 247000] | 40.0 [0, 31100] | 399 [0, 247000] | 3150 [0, 134000] | |
| Max] | | | | | |
| Cytogenetics Risk | | | | | 0.021 |
| Group | | | | | |
| Favorable | 1 (0.735%) | 0 (0%) | 0 (0%) | 1 (2.56%) | |
| Indeterminate | 74 (54.4%) | 26 (70.3%) | 36 (60.0%) | 12 (30.8%) | |
| Unfavorable | 61 (44.9%) | 11 (29.7%) | 24 (40.0%) | 26 (66.7%) | |
| Complex | | | | | 0.017 |
| Cytogenetics | | | | | |
| N | 90 (66.2%) | 29 (78.4%) | 43 (71.7%) | 18 (46.2%) | |
| Y | 46 (33.8%) | 8 (21.6%) | 17 (28.3%) | 21 (53.8%) | |

*p-values indicate significance of clusters 1, 2, and 3 as a group in relation to the indicated clinical characteristics.

### 4. Characterizing Protein Signature Biology: Cluster Networks

Patterns of protein expression were examined across the entire set of 412 proteins that were associated with cluster membership. Proteins were identified in which the median expression was upregulated (log fold change (LFC) above 0) or down-regulated (LFC below 0) relative to the control proteins using the Wilcoxon Test, with a false discovery rate (FDR) adjusted p-value<0.05 and excluded proteins with high expression variability within a cluster. This produced groups of uniformly up- or down-regulated proteins for each cluster (data not shown). By comparing these lists across clusters, cluster-specific signatures were derived where proteins are inversely regulated in one, or both, of the remaining clusters. The list of proteins upregulated in the two unfavorable clusters C2 and C3, relative to the favorable C1 is shown in Table 6. Median relative expression in each cluster represents median expression across the cluster for the correlating protein following the normalization against the median expression of bone-marrow derived CD34+ normal control samples. Positive values are proteins where cluster median is above that of normal and negative, conversely, where cluster median expression is below that of normal.

TABLE 6
Upregulated proteins in either or both unfavorable
prognosis clusters, C2 and C3, relative to C1.

| | Median Relative | Median Relative | Median Relative |
|---|---|---|---|
| Protein Name | Expression C1 | Expression C2 | Expression C3 |
| AKT1_2_3 | −0.0816 | 0.2423 | 0.4989 |
| BAX | 0.1717 | 0.4264 | 0.5654 |
| BECN1 | 0.0679 | 0.1230 | 0.2481 |
| BID | 0.2350 | 0.6270 | 0.9764 |
| CCND3 | 0.0424 | 0.2755 | 0.1980 |
| DUSP6 | −0.3395 | 0.1466 | 0.5973 |
| HEXIM1 | −0.2781 | 0.1433 | 0.3726 |
| HSF1 | −0.6367 | −0.2360 | 0.2390 |
| MAP2K1.pS217_221 | 0.2224 | 0.3659 | 0.3896 |
| MAPK14 | −0.7663 | 0.0831 | 0.3094 |
| MDM2 | −0.1578 | 0.3912 | 0.2865 |
| MYH9.pS1943 | 0.2787 | 1.2843 | 0.5549 |
| PDK1.pS241 | −0.0059 | 0.3099 | 0.4441 |
| PKM2 | 0.5085 | 1.1693 | 1.0882 |
| PREX1 | 0.2019 | 0.5012 | 0.5460 |
| PRKAR1A | 0.1698 | 0.3838 | 0.2525 |
| PRKCB | 0.1318 | 0.4482 | 0.3818 |
| PRKCB.pS660 | 0.0540 | 0.6653 | 0.7097 |
| PTPN11.pY542 | −0.0477 | 0.1808 | 0.1921 |
| RAF1.pS338 | 0.1476 | 0.3756 | 0.3492 |
| RPS6KB1.pT389 | 0.1087 | 0.3141 | 0.2799 |
| SYK | −0.9521 | 0.5038 | 0.6820 |
| TIGAR | 0.2898 | 0.4323 | 0.2755 |
| VAV1 | −0.0228 | 0.6002 | 0.8221 |
| VIM | 0.2150 | 0.7112 | 0.4430 |

The STRING database was queried for literature-based functions of the proteins most differentially up-regulated in C1 (Table 7) to investigate the biology of the favorable prognosis proteome (data not shown). The STRING database does not account for post-translation modifications (PTM), so protein names were queried when proteomics identified a PTM form of a protein. The network of highly-expressed proteins revealed several regulators of cell cycle, growth and proliferation including ADM, PIK3CA, SPARC, and UGT1A1; transcription regulators for coordinating the adaptive response to stress and DNA damage repair including ABL1, FZR1, HIF1A, and NFE2L2; tumor suppressors and proteins critical for regulating growth, autophagy, and apoptosis including ATG4B, BCL2, BIRC2, CASP9, NF2, STK11, TNK1, and TP53; and proteins involved in pluripotent and immune cell differentiation including JAG1, PDCD1, PEA15, and SOX2.

TABLE 7
The 21 most upregulated proteins in C1 signature.
Protein Name

| | | |
|---|---|---|
| ADM | CASP9 | ATG4B |
| PIK3CA | NF2 | BCL2 |
| SPARC | STK11 | BIRC2 |
| UGT1A1 | TNK1 | SOX2 |
| ABL1 | TP53 | ITGA2 |
| FZR1 | JAG1 | NFE2L2 |
| HIF1A | PDCD1 | PEA15 |

In order to investigate the biology of the clusters associated with poor survival outcomes, the same method was used to isolate proteins up-regulated in either or both C2 and C3 (Table 8), which included several proteins and signal transducers involved in pro-inflammatory signaling and immune activation including CBL, HEXIM1, MAPK14, PTPN12, and SYK; promoters of cell growth, survival and proliferation as well as adhesion and cell trafficking including DUSP6, PRKCB, STAT3, TSC2, and VAV1; activators of metabolic pathways including NDRG1, MTOR, and RPS6; and proteins involved in regulation of apoptosis with inhibitors AKT1 and NOL3, as well as the pro-apoptotic BCL-2 family proteins BID and BAX.

TABLE 8
The 23 most upregulated proteins in the C2 and/or C3 signatures
Protein Name

| | | | |
|---|---|---|---|
| CBL | PRKCB | RPS6 | MYH9 |
| HEXIM1 | STAT3 | BAX | PREX1 |
| MAPK14 | TSC2 | BID | BABAM1 |
| PTPN12 | VAV1 | PKM | AKT1 |
| SYK | NDRG1 | RPS6KB1 | NOL3 |
| DUSP6 | MTOR | VASP | |

A person of skill in the art would understand that other biomarkers from Table 3 can be used as alternative systems for determining subject responder status (i.e. determining whether the subject will response to a BCL-2 inhibitor). For example, PRMT1, SPI1, and EIF2AK2 are predictive for distinguishing C1 from C2 (Train C-Index 0.980952381, Val. C-Index 0.989795918, Test C-Index 1, Test Set Overall Accuracy 100%); CASP9, KMT2D, and SMARCB1 are predictive for identifying C1 vs. C3 (Train C-Index 0.996472663, Val. C-Index 1, Test C-Index 1, Test Set Overall Accuracy 100%); and NOTCH1.cle, PRMT1, and CHEK2 are predictive for identifying C2 vs. C3 (Train C-Index 0.997755331, Val. C-Index 0.977777778, Test C-Index 1, Test Set Overall Accuracy 100%).

5. In-Vitro Validation of Relevant Protein Targets that May be Suitable for Therapeutic Inhibition.

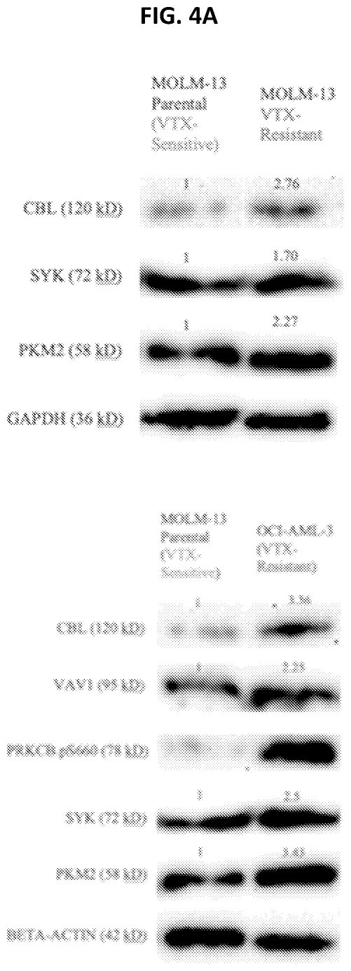

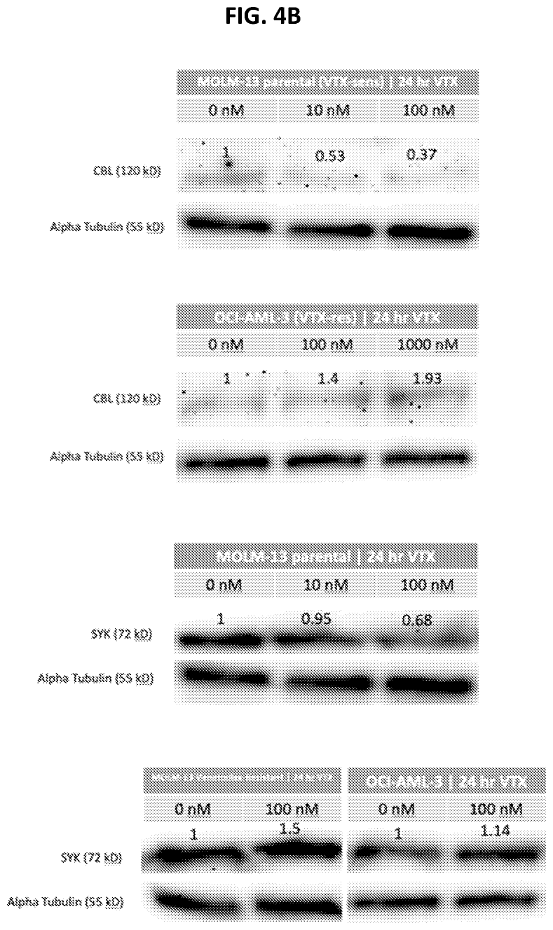

The above findings were validated by looking for similar responses in cell lines in vitro in response to VTX exposure. Two VTX-resistant cell lines, OCI-AML-3 and MOLM-13-VTX-res, and three VTX-sensitive cell lines, MOLM-13-parental, MV4-11, and KG-1, were first validated for their sensitivity to VTX (data not shown). These two VTX-resistant cell lines were characterized by increased baseline expression of potential protein targets that were initially identified from patient cluster analysis, including CBL, SYK, PKM2, VAV1, and PRKCB.pS660, relative to the MOLM-13 VTX-sensitive cell line (FIG. 4A). Of these proteins, CBL and SYK offer promising leads evidenced by their significant downregulation of expression 24 hr post 100 nM VTX treatment in the MOLM-13 parental VTX-sensitive cell line (FIG. 4B). Under the same experimental conditions, CBL was induced in the OCI-3 VTX-resistant cell line, and SYK was induced in both VTX-resistant cell lines MOLM-13-VTX-res and OCI-3. Additional evidence suggests STAT3.pS727 and PKM2 may also be relevant targets to test for efficacy of inhibition. Preliminary results in the MOLM-13 parental VTX-sen cell line showed a reduction in the expression of STAT3.pS727, a phosphoprotein significantly upregulated in the unfavorable cluster relative to the favorable cluster, 24 hours post treatment with VTX. Under identical experimental conditions, MV4-11, another VTX-sen cell line, showed a >50% reduction in the expression of PKM2. These same proteins either remained unchanged or were induced in the OCI-3 VTX-res cell line 24 hours post VTX treatment. These findings taken together with the unbiased hierarchical clustering analysis and the deep learning findings, provide support that SYK, CBL, STAT3.pS727, and PKM2 may be driving resistance to VTX therapy.

### 6. Predicting Cluster Membership for Clinical Use.

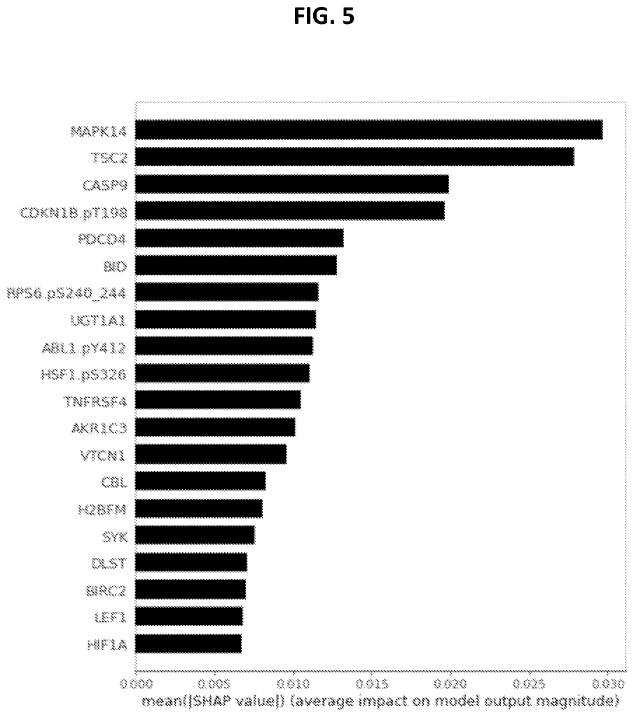

Clinical application of these findings would require a means to rapidly classify which cluster an individual patient belonged to. To facilitate this, small classifier sets were identified, amenable to CLIA certifiable kits, that could do so with high fidelity. Random forests, a predictive machine learning algorithm, were employed to identify the smallest number of proteins with the highest accuracy to predict cluster membership. For each of following deep learning models, patients were split into development and test sets using an 80/20 split, respectively. Model hyperparameters, including the number of trees used in the forest, the maximum depth of each tree, and the minimum number of proportion of samples in a leaf, were optimized using grid search. Binary classification models were generated for C1 vs C2, C1 vs C3, and C2 vs C3. Across the broader set of 412 assayed proteins, random forests returned model-specific sets of proteins with high SHapley Additive exPlanations (SHAP) values correlated to having the strongest contribution to the predictive value of the model (FIG. 5, Table 9). Analysis of these model-specific protein sets identified five proteins—CDKN1B.pT198, MSH6, ASH2L, MDM2.pS166, and ITGA2—as strong contributors across all three models. A random forest model developed with these five proteins as a classifier had strong discriminatory power with a very high c-index of 0.965 and predicted C1 vs C2 and C1 vs C3 with 95% and 100% accuracy, relatively. See Table 10 for parameters of machine learning model (Python package: sklearn.ensemble.RandomForestClassifier). The model predicted C2 vs C3 membership, the unfavorable outcome clusters, with 76% accuracy.

TABLE 9
Set of proteins with strongest contribution to predictive
value for binary classification models.
Protein Name

| | |
|---|---|
| MAPK14 | TNFRSF4 |
| TSC2 | AKR1C3 |
| CASP9 | VTCN1 |
| CDKN1B.pT198 | CBL |
| PDCD4 | H2BFM |
| BID | SYK |
| RPS6.pS240_244 | DLST |
| UGT1A1 | BIRC2 |
| ABL1.pY412 | LEF1 |
| HSF1.pS326 | HIF1A |

TABLE 10
Machine Learning Model Parameters.

| | |
|---|---|
| Model: | Proteins inputs to model: CDKN1B.pT198, MSH6, ASH2L, MDM2.pS166, ITGA2 |
| C1 vs C2 | Best hyperparameters: |
| | {‘n_estimators’: 500, ‘max_depth’: 10, ‘min_samples_leaf’: 10, ‘random_state’: 9} |
| | Train C-Index: 0.969017094017094 |
| | Val C-Index: 0.9230769230769231 |
| | Test C-Index: 0.9897959183673469 |
| Model: | Proteins inputs to model: CDKN1B.pT198, MSH6, ASH2L, MDM2.pS166, ITGA2 |
| C1 vs C3 | Best hyperparameters: |
| | {‘n_estimators’: 500, ‘max_depth’: 10, ‘min_samples leaf’: 10, ‘random_state’: 9} |
| | Train C-Index: 1.0 |
| | Val C-Index: 1.0 |
| | Test C-Index: 1.0 |
| Model: | Proteins inputs to model: CDKN1B.pT198, MSH6, ASH2L, MDM2.pS166, ITGA2 |
| | Best hyperparameters: |
| C2 vs C3 | {‘n_estimators’: 500, ‘max_depth’: 1, ‘min_samples_leaf’: 8, ‘random_state’: 18} |
| | Train C-Index: 0.9352869352869353 |
| | Val C-Index: 0.9519230769230769 |
| | Test C-Index: 0.8636363636363636 |

### REFERENCES RELATING TO EXAMPLES

1. Juliusson, G., et al., Prevalence and characteristics of survivors from acute myeloid leukemia in Sweden. Leukemia, 2017. 31(3): p. 728-731.
2. Kale, J., et al., BCL-2 family proteins: changing partners in the dance towards death. Cell Death Differ, 2018. 25(1): p. 65-80.
3. DiNardo, C. D., et al., Venetoclax combined with decitabine or azacitidine in treatment-naive, elderly patients with acute myeloid leukemia. Blood, 2019. 133(1): p. 7-17.
4. Wei, A. H., et al., Venetoclax Combined With Low-Dose Cytarabine for Previously Untreated Patients With Acute Myeloid Leukemia: Results From a Phase Ib/II Study. J Clin Oncol, 2019. 37(15): p. 1277-1284.
5. Bose, P., et al., Pathways and mechanisms of venetoclax resistance. Leuk Lymphoma, 2017. 58(9): p. 1-17.
6. DiNardo, C. D., et al., Molecular patterns of response and treatment failure after frontline venetoclax combinations in older patients with AML. Blood, 2020. 135(11): p. 791-803.
7. Tibes, R., et al., Reverse phase protein array: validation of a novel proteomic technology and utility for analysis of primary leukemia specimens and hematopoietic stem cells. Mol Cancer Ther, 2006. 5(10): p. 2512-21.
8. Kornblau, S. M. and K. R. Coombes, Use of reverse phase protein microarrays to study protein expression in leukemia: technical and methodological lessons learned. Methods Mol Biol, 2011. 785: p. 141-55.
9. Kornblau, S. M., et al., Comparative analysis of the effects of sample source and test methodology on the assessment of protein expression in acute myelogenous leukemia. Leukemia, 2005. 19(9): p. 1550-7.
10. Hu, C. W., et al., A quantitative analysis of heterogeneities and hallmarks in acute myelogenous leukaemia. Nat Biomed Eng, 2019. 3(11): p. 889-901.
11. Hu J, et al., Non-parametric quantification of protein lysate arrays. Bioinformatics, 2007. 23(15): p. 1986-94.
12. Therneau, T., A Package for Survival Analysis in R, in R package version 3.2-3. 2020. p. url: https://CRAN.R-project.org/package=survival.
13. Therneau, T. G., PM, Modeling Survival Data: Extending the Cox Model. 2000, New York: Springer.
14. Hu, C. W., et al., Progeny Clustering: A Method to Identify Biological Phenotypes. Sci Rep, 2015. 5: p. 12894.
15. Ruvolo, P. P., et al., May, Phosphorylation of Bcl2 and regulation of apoptosis. Leukemia, 2001. 15(4): p. 515-22.
16. Zhou, J.-d., et al., BCL2 overexpression: clinical implication and biological insights in acute myeloid leukemia. Diagnostic Pathology, 2019. 14(1): p. 68.
17. Wei, Y., et al., Targeting Bcl-2 Proteins in Acute Myeloid Leukemia. Frontiers in Oncology, 2020. 10.
18. Guieze, R., et al., Mitochondrial Reprogramming Underlies Resistance to BCL-2 Inhibition in Lymphoid Malignancies. Cancer Cell, 2019. 36(4): p. 369-384 e13.
19. Hoff, F. W, et al. Antibody Screening. Adv Exp Med Biol. 2019; 1188:149-63.

The above description of example embodiments of the present disclosure has been presented for the purposes of illustration and description. It is not intended to be exhaustive or to limit the disclosure to the precise form described, and many modifications and variations are possible in light of the teaching above.

A recitation of “a”, “an” or “the” is intended to mean “one or more” unless specifically indicated to the contrary. The use of “or” is intended to mean an “inclusive or,” and not an “exclusive or” unless specifically indicated to the contrary. Reference to a “first” component does not necessarily require that a second component be provided. Moreover, reference to a “first” or a “second” component does not limit the referenced component to a particular location unless expressly stated. The term “based on” is intended to mean “based at least in part on.”

The terms “about” and “approximately” as used herein shall generally mean an acceptable degree of error for the quantity measured given the nature or precision of the measurements. Typically, exemplary degrees of error are within 20 percent (%), preferably within 10%, and more preferably within 5% of a given value or range of values. Any reference to “about X” specifically indicates at least the values X, 0.8X, 0.81X, 0.82X, 0.83X, 0.84X, 0.85X, 0.86X, 0.87X, 0.88X, 0.89X, 0.9X, 0.91X, 0.92X, 0.93X, 0.94X, 0.95X, 0.96X, 0.97X, 0.98X, 0.99X, 1.01X, 1.02X, 1.03X, 1.04X, 1.05X, 1.06X, 1.07X, 1.08X, 1.09X, 1.1X, 1.11X, 1.12X, 1.13X, 1.14X, 1.15X, 1.16X, 1.17X, 1.18X, 1.19X, and 1.2X. Thus, “about X” is intended to teach and provide written description support for a claim limitation of, e.g., “0.98X.”

All patents, patent applications, publications, and descriptions mentioned herein are incorporated by reference in their entirety for all purposes. None is admitted to be prior art. Where a conflict exists between the instant application and a reference provided herein, the instant application shall dominate.

When a group of substituents is disclosed herein, it is understood that all individual members of those groups and all subgroups and classes that can be formed using the substituents are disclosed separately. When a Markush group or other grouping is used herein, all individual members of the group and all combinations and subcombinations possible of the group are intended to be individually included in the disclosure. As used herein, “and/or” means that one, all, or any combination of items in a list separated by “and/or” are included in the list; for example “1, 2 and/or 3” is equivalent to “‘1’ or ‘2’ or ‘3’ or ‘1 and 2’ or ‘1 and 3’ or ‘2 and 3’ or ‘1, 2 and 3’”. Whenever a range is given in the specification, for example, a temperature range, a time range, or a composition range, all intermediate ranges and subranges, as well as all individual values included in the ranges given are intended to be included in the disclosure.

What is claimed is:

1. A method of determining the likelihood that a subject with acute myeloid leukemia (AML) will respond to a therapy comprising the administration of a BCL-2 inhibitor, the method comprising detecting protein levels of one or more AML therapy response biomarkers in a biological sample comprising AML cells obtained from the subject, wherein the one or more AML therapy response biomarkers comprises one or more biomarkers listed in Table 3.

2. The method of claim 1, wherein the one or more AML therapy response biomarkers comprise one or more biomarkers listed in Table 9.

3. The method of claim 1, wherein 2, 3, 4, 5, 6, 7, 8, 9, 10, or more AML therapy response biomarkers are detected in the biological sample.

4. The method of claim 1, wherein the AML therapy response biomarkers comprise one or more of CDKN1B.pT198, MSH6, ASH2L, MDM2.pS166, or ITGA2.

5. The method of claim 1, wherein the method further comprises calculating a response score based on the detected protein levels of the AML therapy response biomarkers, wherein the response score corresponds to the likelihood that the subject will respond to the therapy.

6. The method of claim 1, wherein the subject is determined to have a high likelihood of responding to the therapy.

7. The method of claim 6, further comprising administering the therapy to the subject.

8. The method of claim 1, wherein the subject is determined to have a low likelihood of responding to the therapy.

9. The method of claim 8, further comprising administering an alternative therapy to the subject that does not comprise the BCL-2 inhibitor.

10. The method of claim 1, wherein the BCL-2 inhibitor is venetoclax.

11. The method of claim 1, wherein a response to the therapy comprises an increase in remission duration.

12. The method of claim 1, wherein a response to the therapy comprises an increase in overall survival.

13. The method of claim 1, wherein the biological sample is a blood sample or a bone marrow sample.

14. The method of claim 1, wherein the protein levels of the one or more AML therapy response biomarkers are measured using one or more antibodies that specifically bind to the one or more biomarkers.

15. A method of generating a report containing information on the likelihood that a subject with AML will respond to a therapy comprising a BCL-2 inhibitor, comprising:
detecting in a biological sample obtained from the subject the protein levels of one or more AML therapy response biomarkers; and,
generating the report,
wherein the one or more AML therapy response biomarkers comprise one or more biomarkers listed in Table 3,
and wherein the report is useful for determining the likelihood that the subject will respond to the therapy.

16. The method of claim 15, wherein the one or more AML therapy response biomarkers comprise one or more biomarkers listed in Table 9.

17. The method of claim 15, wherein the one or more AML therapy response biomarkers comprise at least one of CDKN1B.pT198, MSH6, ASH2L, MDM2.pS166, or ITGA2.

18. A system for determining the likelihood that a subject with AML will respond to a therapy comprising a BCL-2 inhibitor, comprising a station for analyzing a biological sample comprising AML cells obtained from the subject to measure protein levels of one or more AML therapy response biomarkers in the sample, wherein the one or more AML therapy response biomarkers comprise one or more biomarkers listed in Table 3.

19. The system of claim 18, wherein the one or more AML therapy response biomarkers comprise one or more biomarkers listed in Table 9.

20. The method of claim 18, wherein the one or more AML therapy response biomarkers comprise at least one of CDKN1B.pT198, MSH6, ASH2L, MDM2.pS166, or ITGA2.

21. The system of claim 18, further comprising a station for generating a report containing information on results of the analyzing.

22. A method of treating a subject with acute myeloid leukemia (AML), the method comprising administering to the subject a therapeutically effective amount of a BCL-2 inhibitor to a subject having differential levels of one or more AML response biomarkers in a biological sample from the subject as compared to a control, wherein the one or more biomarkers comprise at least one of CDKN1B.pT198, MSH6, ASH2L, MDM2.pS166, or ITGA2, and/or any one or more of biomarkers listed in Table 3 or Table 9.

23. A method of treating a subject with acute myeloid leukemia (AML), the method comprising administering to the subject a therapeutically effective amount of a BCL-2 inhibitor, wherein the subject has been identified as a likely responder to the BCL-2 inhibitor based on a detection of the protein levels of one or more AML therapy response biomarkers in a biological sample comprising AML cells obtained from the subject, wherein the identification of the subject as a likely responder is based on a difference in the one or more protein levels relative to protein levels of the one or more AML therapy response biomarkers in a biological sample from a healthy individual without AML, and wherein the one or more AML therapy response biomarkers comprise one or more biomarkers listed in Table 3.

24. The method of claim 22 or 23, wherein the one or more AML therapy response biomarkers comprise one or more biomarkers listed in Table 9.

25. The method of claim 22 or 23, wherein 2, 3, 4, 5, 6, 7, 8, 9, 10, or more AML therapy response biomarkers are detected in the biological sample.

26. The method of claim 23, wherein the AML therapy response biomarkers comprise at least one of CDKN1B.pT198, MSH6, ASH2L, MDM2.pS166, or ITGA2.

27. The method of claim 23, wherein the identification of the subject as a likely responder comprises the calculation of a response score based on the detected protein levels of the AML therapy response biomarkers, wherein the response score corresponds to the likelihood that the subject will respond to the therapy.

28. The method of claim 22 or 23, wherein the BCL-2 inhibitor is venetoclax.

29. The method of claim 22 or 23, wherein the administration of the therapy leads to an increase in remission duration.

30. The method of claim 22 or 23, wherein the administration of the therapy leads to an increase in overall survival.

31. The method of claim 22 or 23, wherein the biological sample is a blood sample or a bone marrow sample.

32. The method of claim 27, wherein the protein levels of the one or more AML therapy response biomarkers are measured using one or more antibodies that specifically bind to the one or more biomarkers.

33. The method of claim 32, wherein the protein levels are measured using an ELISA assay.

34. A method of generating a report containing information on the likelihood that a subject with AML will not respond to a therapy comprising a BCL-2 inhibitor, comprising:
detecting in a biological sample obtained from the subject the protein levels of one or more AML therapy response biomarkers; and,
generating the report,
wherein the one or more AML therapy response biomarkers comprise one or more biomarkers listed in Table 3,
and wherein the report is useful for determining the likelihood that the subject will not respond to the therapy.

35. The method of claim 34, wherein the AML therapy response biomarkers comprise one or more biomarkers listed in Table 9.

36. The method of claim 31, wherein the one or more AML therapy response biomarkers comprise at least one of CDKN1B.pT198, MSH6, ASH2L, MDM2.pS166, or ITGA2.
